SEQUENCE LISTING (26 sequences)

SEQ ID NO: 1 - RNA, 49 nt, Lentivirus humimdef
  source 1..49: mol_type = other RNA
gaccaccccaaaaatgaaggggactaaactttttttttttttgaagcac

SEQ ID NO: 2 - RNA, 50 nt, Lentivirus humimdef
  source 1..50: mol_type = other RNA
gaccaccccaaaaatgaaggggactaaaacctgcttatatgcaggatctg

SEQ ID NO: 3 - RNA, 50 nt, Lentivirus humimdef
  source 1..50: mol_type = other RNA
gaccaccccaaaaatgaaggggactaaaacccagagagacccagtacagg

SEQ ID NO: 4 - RNA, 50 nt, Lentivirus humimdef
  source 1..50: mol_type = other RNA
gaccaccccaaaaatgaaggggactaaaaccctgccataggagatgccta

SEQ ID NO: 5 - RNA, 50 nt, Lentivirus humimdef
  source 1..50: mol_type = other RNA
gaccaccccaaaaatgaaggggactaaaacgtctccgcttcttcctgcca

SEQ ID NO: 6 - RNA, 50 nt, Lentivirus humimdef
  source 1..50: mol_type = other RNA
gaccaccccaaaaatgaaggggactaaaacagcttgatgagtctgactgt

SEQ ID NO: 7 - RNA, 50 nt, Lentivirus humimdef
  source 1..50: mol_type = other RNA
gaccaccccaaaaatgaaggggactaaaacttccttcgggcctgtcgggt

SEQ ID NO: 8 - RNA, 50 nt, Lentivirus humimdef
  source 1..50: mol_type = other RNA
gaccaccccaaaaatgaaggggactaaaacggatctgtctctgtctctct

SEQ ID NO: 9 - RNA, 30 nt, Lentivirus humimdef
  source 1..30: mol_type = other RNA
gaccaccccaaaaatgaaggggactaaaac

SEQ ID NO: 10 - protein, 1177 aa, Leptotrichia wadei
MKITKIDGVSHYKKQDKGILKKKWKDLDERKQREKIEARYNKQIESKIYKEFFRLKNKKRIEKEEDQNIKSLYFFIKELYLNEKNEEWELKNINLEILDDKERVIKGYKFKEDVYFFKEGYKEYYLRILFNNLIEKVQNENREKVRKNKEFLDLKEIFKKYKNRKIDLLLKSINNNKINLEYKKENVNEEIYGINPTNDREMTFYELLKEIIEKKDEQKSILEEKLDNFDITNFLENIEKIFNEETEINIIKGKVLNELREYIKEKEENNSDNKLKQIYNLELKKYIENNFSYKKQKSKSKNGKNDYLYLNFLKKIMFIEEVDEKKEINKEKFKNKINSNFKNLFVQHILDYGKLLYYKENDEYIKNTGQLETKDLEYIKTKETLIRKMAVLVSFAANSYYNLFGRVSGDILGTEVVKSSKTNVIKVGSHIFKEKMLNYFFDFEIFDANKIVEILESISYSIYNVRNGVGHFNKLILGKYKKKDINTNKRIEEDLNNNEEIKGYFIKKRGEIERKVKEKFLSNNLQYYYSKEKIENYFEVYEFEILKRKIPFAPNFKRIIKKGEDLFNNKNNKKYEYFKNFDKNSAEEKKEFLKTRNFLLKELYYNNFYKEFLSKKEEFEKIVLEVKEEKKSRGNINNKKSGVSFQSIDDYDTKINISDYIASIHKKEMERVEKYNEEKQKDTAKYIRDFVEEIFLTGFINYLEKDKRLHFLKEEFSILCNNNNNVVDFNININEEKIKEFLKENDSKTLNLYLFFNMIDSKRISEFRNELVKYKQFTKKRLDEEKEFLGIKIELYETLIEFVILTREKLDTKKSEEIDAWLVDKLYVKDSNEYKEYEEILKLFVDEKILSSKEAPYYATDNKTPILLSNFEKTRKYGTQSFLSEIQSNYKYSKVEKENIEDYNKKEEIEQKKKSNIEKLQDLKVELHKKWEQNKITEKEIEKYNNTTRKINEYNYLKNKEELQNVYLLHEMLSDLLARNVAFFNKWERDFKFIVIAIKQFLRENDKEKVNEFLNPPDNSKGKKVYFSVSKYKNTVENIDGIHKNFMNLIFLNNKFMNRKIDKMNCAIWVYFRNYIAHFLHLHTKNEKISLISQMNLLIKLFSYDKKVQNHILKSTKTLLEKYNIQINFEISNDKNEVFKYKIKNRLYSKKGKMLGKNNKLENEFLENVKAMLEYSE

SEQ ID NO: 11 - protein, 1285 aa, Herbinix hemicellulosilytica
MKLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVARYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 12 - protein, 1159 aa, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 13 - protein, 1120 aa, Leptotrichia seeligeri
MWISIKTLIHHLGVLFFCDYMYNRREKKIIEVKTMRITKVEVDRKKVLISRDKNGGKLVYENEMQDNTEQIMHHKKSSFYKSVVNKTICRPEQKQMKKLVHGLLQENSQEKIKVSDVTKLNISNFLNHRFKKSLYYFPENSPDKSEEYRIEINLSQLLEDSLKKQQGTFICWESFSKDMELYINWAENYISSKTKLIKKSIRNNRIQSTESRSGQLMDRYMKDILNKNKPFDIQSVSEKYQLEKLTSALKATFKEAKKNDKEINYKLKSTLQNHERQIIEELKENSELNQFNIEIRKHLETYFPIKKTNRKVGDIRNLEIGEIQKIVNHRLKNKIVQRILQEGKLASYEIESTVNSNSLQKIKIEEAFALKFINACLFASNNLRNMVYPVCKKDILMIGEFKNSFKEIKHKKFIRQWSQFFSQEITVDDIELASWGLRGAIAPIRNEIIHLKKHSWKKFFNNPTFKVKKSKIINGKTKDVTSEFLYKETLFKDYFYSELDSVPELIINKMESSKILDYYSSDQLNQVFTIPNFELSLLTSAVPFAPSFKRVYLKGFDYQNQDEAQPDYNLKLNIYNEKAFNSEAFQAQYSLFKMVYYQVFLPQFTTNNDLFKSSVDFILTLNKERKGYAKAFQDIRKMNKDEKPSEYMSYIQSQLMLYQKKQEEKEKINHFEKFINQVFIKGFNSFIEKNRLTYICHPTKNTVPENDNIEIPFHTDMDDSNIAFWLMCKLLDAKQLSELRNEMIKFSCSLQSTEEISTFTKAREVIGLALLNGEKGCNDWKELFDDKEAWKKNMSLYVSEELLQSLPYTQEDGQTPVINRSIDLVKKYGTETILEKLFSSSDDYKVSAKDIAKLHEYDVTEKIAQQESLHKQWIEKPGLARDSAWTKKYQNVINDISNYQWAKTKVELTQVRHLHQLTIDLLSRLAGYMSIADRDFQFSSNYILERENSEYRVTSWILLSENKNKNKYNDYELYNLKNASIKVSSKNDPQLKVDLKQLRLTLEYLELFDNRLKEKRNNISHFNYLNGQLGNSILELFDDARDVLSYDRKLKNAVSKSLKEILSSHGMEVTFKPLYQTNHHLKIDKLQPKKIHHLGEKSTVSSNQVSNEYCQLVRTLLTMK

SEQ ID NO: 14 - protein, 1154 aa, Paludibacter propionicigenes
MRVSKVKVKDGGKDKMVLVHRKTTGAQLVYSGQPVSNETSNILPEKKRQSFDLSTLNKTIIKFDTAKKQKLNVDQYKIVEKIFKYPKQELPKQIKAEEILPFLNHKFQEPVKYWKNGKEESFNLTLLIVEAVQAQDKRKLQPYYDWKTWYIQTKSDLLKKSIENNRIDLTENLSKRKKALLAWETEFTASGSIDLTHYHKVYMTDVLCKMLQDVKPLTDDKGKINTNAYHRGLKKALQNHQPAIFGTREVPNEANRADNQLSIYHLEVVKYLEHYFPIKTSKRRNTADDIAHYLKAQTLKTTIEKQLVNAIRANIIQQGKTNHHELKADTTSNDLIRIKTNEAFVLNLTGTCAFAANNIRNMVDNEQTNDILGKGDFIKSLLKDNTNSQLYSFFFGEGLSTNKAEKETQLWGIRGAVQQIRNNVNHYKKDALKTVFNISNFENPTITDPKQQTNYADTIYKARFINELEKIPEAFAQQLKTGGAVSYYTIENLKSLLTTFQFSLCRSTIPFAPGFKKVFNGGINYQNAKQDESFYELMLEQYLRKENFAEESYNARYFMLKLIYNNLFLPGFTTDRKAFADSVGFVQMQNKKQAEKVNPRKKEAYAFEAVRPMTAADSIADYMAYVQSELMQEQNKKEEKVAEETRINFEKFVLQVFIKGFDSFLRAKEFDFVQMPQPQLTATASNQQKADKLNQLEASITADCKLTPQYAKADDATHIAFYVFCKLLDAAHLSNLRNELIKFRESVNEFKFHHLLEIIEICLLSADVVPTDYRDLYSSEADCLARLRPFIEQGADITNWSDLFVQSDKHSPVIHANIELSVKYGTTKLLEQIINKDTQFKTTEANFTAWNTAQKSIEQLIKQREDHHEQWVKAKNADDKEKQERKREKSNFAQKFIEKHGDDYLDICDYINTYNWLDNKMHFVHLNRLHGLTIELLGRMAGFVALFDRDFQFFDEQQIADEFKLHGFVNLHSIDKKLNEVPTKKIKEIYDIRNKIIQINGNKINESVRANLIQFISSKRNYYNNAFLHVSNDEIKEKQMYDIRNHIAHFNYLTKDAADFSLIDLINELRELLHYDRKLKNAVSKAFIDLFDKHGMILKLKLNADHKLKVESLEPKKIYHLGSSAKDKPEYQYCTNQVMMAYCNMCRSLLEMKK

SEQ ID NO: 15 - protein, 1437 aa, Lachnospiraceae bacterium
MKISKVREENRGAKLTVNAKTAVVSENRSQEGILYNDPSRYGKSRKNDEDRDRYIESRLKSSGKLYRIFNEDKNKRETDELQWFLSEIVKKINRRNGLVLSDMLSVDDRAFEKAFEKYAELSYTNRRNKVSGSPAFETCGVDAATAERLKGIISETNFINRIKNNIDNKVSEDIIDRIIAKYLKKSLCRERVKRGLKKLLMNAFDLPYSDPDIDVQRDFIDYVLEDFYHVRAKSQVSRSIKNMNMPVQPEGDGKFAITVSKGGTESGNKRSAEKEAFKKFLSDYASLDERVRDDMLRRMRRLVVLYFYGSDDSKLSDVNEKFDVWEDHAARRVDNREFIKLPLENKLANGKTDKDAERIRKNTVKELYRNQNIGCYRQAVKAVEEDNNGRYFDDKMLNMFFIHRIEYGVEKIYANLKQVTEFKARTGYLSEKIWKDLINYISIKYIAMGKAVYNYAMDELNASDKKEIELGKISEEYLSGISSFDYELIKAEEMLQRETAVYVAFAARHLSSQTVELDSENSDFLLLKPKGTMDKNDKNKLASNNILNFLKDKETLRDTILQYFGGHSLWTDFPFDKYLAGGKDDVDFLTDLKDVIYSMRNDSFHYATENHNNGKWNKELISAMFEHETERMTVVMKDKFYSNNLPMFYKNDDLKKLLIDLYKDNVERASQVPSFNKVFVRKNFPALVRDKDNLGIELDLKADADKGENELKFYNALYYMFKEIYYNAFLNDKNVRERFITKATKVADNYDRNKERNLKDRIKSAGSDEKKKLREQLQNYIAENDFGQRIKNIVQVNPDYTLAQICQLIMTEYNQQNNGCMQKKSAARKDINKDSYQHYKMLLLVNLRKAFLEFIKENYAFVLKPYKHDLCDKADFVPDFAKYVKPYAGLISRVAGSSELQKWYIVSRFLSPAQANHMLGFLHSYKQYVWDIYRRASETGTEINHSIAEDKIAGVDITDVDAVIDLSVKLCGTISSEISDYFKDDEVYAEYISSYLDFEYDGGNYKDSLNRFCNSDAVNDQKVALYYDGEHPKLNRNIILSKLYGERRFLEKITDRVSRSDIVEYYKLKKETSQYQTKGIFDSEDEQKNIKKFQEMKNIVEFRDLMDYSEIADELQGQLINWIYLRERDLMNFQLGYHYACLNNDSNKQATYVTLDYQGKKNRKINGAILYQICAMYINGLPLYYVDKDSSEWTVSDGKESTGAKIGEFYRYAKSFENTSDCYASGLEIFENISEHDNITELRNYIEHFRYYSSFDRSFLGIYSEVFDRFFTYDLKYRKNVPTILYNILLQHFVNVRFEFVSGKKMIGIDKKDRKIAKEKECARITIREKNGVYSEQFTYKLKNGTVYVDARDKRYLQSIIRLLFYPEKVNMDEMIEVKEKKKPSDNNTGKGYSKRDRQQDRKEYDKYKEKKKKEGNFLSGMGGNINWDEINAQLKN

SEQ ID NO: 16 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 17 - protein, 1159 aa, Leptotrichia buccalis; identical to SEQ ID NO: 12

SEQ ID NO: 18 - RNA, 50 nt, Hepacivirus C
  source 1..50: mol_type = other RNA
gaccaccccaaaaatgaaggggactaaaacgtgtactcaccggttccgca

SEQ ID NO: 19 - RNA, 50 nt, Hepacivirus C
  source 1..50: mol_type = other RNA
gaccaccccaaaaatgaaggggactaaaacccctatcaggcagtaccaca

SEQ ID NO: 20 - RNA, 50 nt, Hepacivirus C
  source 1..50: mol_type = other RNA
gaccaccccaaaaatgaaggggactaaaacaccgggtaggttccctgttg

SEQ ID NO: 21 - RNA, 50 nt, Hepacivirus C
  source 1..50: mol_type = other RNA
gaccaccccaaaaatgaaggggactaaaacgggcgaccagttcatcatca

SEQ ID NO: 22 - RNA, 50 nt, Hepacivirus C
  source 1..50: mol_type = other RNA
gaccaccccaaaaatgaaggggactaaaacgacgatgaccttcttctcca

SEQ ID NO: 23 - RNA, 50 nt, Hepacivirus C
  source 1..50: mol_type = other RNA
gaccaccccaaaaatgaaggggactaaaacttccactgccagttggagca

SEQ ID NO: 24 - RNA, 50 nt, Hepacivirus C
  source 1..50: mol_type = other RNA
gaccaccccaaaaatgaaggggactaaaacgttcatccattggaccgcgc

SEQ ID NO: 25 - RNA, 50 nt, Hepacivirus C
  source 1..50: mol_type = other RNA
gaccaccccaaaaatgaaggggactaaaacggctcgagaaagtccagaac

SEQ ID NO: 26 - RNA, 50 nt, Hepacivirus C
  source 1..50: mol_type = other RNA
gaccaccccaaaaatgaaggggactaaaactctgcagagagaccagttac

(19) United States
(12) Patent Application Publication
(10) Pub. No.: US 2025/0382658 A1
(43) Pub. Date: Dec. 18, 2025

(54) HIV OR HCV DETECTION WITH CRISPR-CAS13A

(71) Applicants: The J. David Gladstone Institutes, a testamentary trust established under the Will of J. David Glad, San Francisco, CA (US); The Regents of the University of California, Oakand, CA (US)

(72) Inventors: Parinaz Fozouni et al., San Francisco, CA (US); Melanie Ott, Mill Valley, CA (US); Jennifer A. Doudna, Oakland, CA (US)

(21) Appl. No.: 19/017,536

(22) Filed: Jan. 11, 2025

Related U.S. Application Data
(63) Continuation of application No. 17/273,752, filed on Mar. 5, 2021, now Pat. No. 12,215,377.
(60) Provisional application No. 62/728,329, filed on Sep. 7, 2018.

(51) Int. Cl. C12Q 1/6806; C12N 9/22; C12N 15/11; C12Q 1/6888; C12Q 1/70

(52) U.S. Cl. CPC C12Q 1/6806; C12N 9/22; C12N 15/11; C12Q 1/6888; C12Q 1/703; C12N 2310/20; C12Q 2600/158

(57) ABSTRACT

The present disclosure relates to methods using CRISPR-Cas13a enzyme, complexed with HIV or HCV crRNAs to specifically and sensitively detect and quantify the presence of HIV or HCV RNA in a sample. These methods can be used to diagnose HIV or HCV infection, quantify the concentration of HIV or HCV RNA present in a sample, identify the presence of different HIV or HCV splice variants, subtypes, or mutations, and to monitor reactivation of HIV or HCV transcription.

### PRIORITY APPLICATIONS

This application is a continuation of U.S. patent application Ser. No. 17/273,752, filed Mar. 5, 2021, which is a U.S. National Stage Filing under 35 U.S.C. 371 from International Patent Application Serial No. PCT/US2019/049954, filed Sep. 6, 2019, published on Mar. 12, 2020 as WO 2020/051452 A2, which application claims benefit of priority to the filing date of U.S. Provisional Application Ser. No. 62/728,329, filed Sep. 7, 2018, the contents of which are specifically incorporated herein by reference in their entireties.

### GOVERNMENT FUNDING

This invention was made with government support under R61 AI140465 awarded by the National Institutes of Health. The government has certain rights in the invention.

### INCORPORATION BY REFERENCE OF SEQUENCE LISTING

This application contains a Sequence Listing which has been submitted electronically in ST26 format and hereby incorporated by reference in its entirety. Said ST26 file, created on Sep. 4, 2025, is named 3730027US2.xml and is 34,281 bytes in size.

### BACKGROUND

There are approximately 36.7 million people worldwide living with HIV/AIDS. It is estimated that 1.8 million new cases of HIV infection occurred in 2016, with about 5,000 new infections per day. In addition, currently only about 70% of people with HIV know of their positive status.

Accurate detection of acute and chronic HIV infections remains a significant challenge. Acute HIV-1 infection is the phase of HIV-1 disease immediately after infection and is characterized by detectable HIV-1 viremia or p24 antigen but having a yet undetectable antibody response. To date, all HIV self-testing products are serology-based, detecting antibodies to HIV-1 four weeks to three months, on average, after exposure. Some assays combine antibody detection with p24 antigen measurements, which allows HIV-1 detection as early as about 18 days after exposure but can have a detection window as large as between about 18 to 90 days. This remains a major detractor against HIV-1 self-testing, as self-tests with such a large window can provide false-negative results during acute infection, false reassurance, and can promote intercourse between discordant partners at the time of highest infectivity.

Early testing is currently laboratory-based and directed against nucleic acids of the viral genome (NAT), reliably detecting HIV-1 RNA about one week after exposure. Frequent NAT-based testing is required after treatment interruptions of chronically infected individuals who all present antibodies and are thus limited to NAT-based, not antibody-based, strategies and subject to frequent laboratory visits. Detection of viral RNA is the gold standard of HIV-1 diagnostics, but current state-of-the-art testing requires laboratory access and cannot be performed at home.

The United Nations has set targets to diagnose 90% of all people living with HIV-1 by 2020, however the World Health Organization (WHO) estimates that only 70% of people infected with HIV-1 currently know their HIV-1 status. As such, there is a critical need to develop new technologies for sensitive, easy-to-handle detection of HIV-1 that allows for frequent at-home testing.

The hepatitis C virus (which may be abbreviated as “HCV” hereinafter) was discovered as a major causative virus of non-A and non-B hepatitis. HCV is a single-stranded (+) RNA virus having a genome length of approximately 9.6 kb, in which the genome encodes a precursor protein that is divided into 10 types of virus protein (i.e., Core, E1, E2, p7, NS2, NS3, NS4A, NS4B, NS5A, and NS5B proteins) via post-translational cleavage by signal peptidase from host or proteases from HCV.

HCV is transmitted from human to human via blood, causing chronic hepatitis among about 60% to 80% of infected persons. When hepatitis is left without any appropriate treatment, it is known to cause cirrhosis or liver cancer within about 20 to 30 years after infection. Therefore, early detection of HCV infection for prevention of the onset of chronic hepatitis and early treatment of the disease after the onset thereof are desired.

### SUMMARY

In some respects, this disclosure provides methods that include (a) incubating a sample containing RNA with a Cas13a protein and at least one CRISPR guide RNA (crRNA) for a period of time sufficient to form one or more HIV or HCV RNA cleavage product(s); and (b) detecting level(s) of HIV or HCV RNA cleavage product(s) with a detector, wherein the RNA is not reverse transcribed prior to the detecting step. Such methods are useful for detecting whether the sample contains one or more copies of an HIV or HCV RNA. The methods are also useful for detecting the absence of an HIV or HCV infection.

In some aspects the disclosure provides methods for quantifying HIV or HCV RNA concentration in a sample comprising (a) incubating a sample containing RNA with a Cas13a protein and at least one CRISPR guide RNA (crRNA) for a period of time sufficient to form one or more HIV or HCV RNA cleavage product(s); and (b) analyzing HIV or HCV RNA cleavage product quantity or concentration with a detector, wherein the RNA is not reverse transcribed prior to the detecting step.

In some aspects the disclosure provides methods for identifying the presence or absence of HIV or HCV splice variants and/or mutations in HIV or HCV RNA in a sample comprising (a) incubating a mixture comprising a sample containing RNA, a Cas13a protein, and at least one CRISPR guide RNA (crRNA) for a period of time sufficient to form one or more HIV or HCV RNA cleavage product(s); and (b) detecting any HIV or HCV splice variants and/or mutations in HIV or HCV RNA by analyzing any HIV or HCV RNA cleavage product(s) with a detector, wherein the RNA is not reverse transcribed prior to the detecting step.

In some aspects the disclosure provides methods for monitoring reactivation of HIV or HCV transcription comprising (a) incubating a sample containing RNA with a Cas13a protein and at least one CRISPR guide RNA (crRNA) for a period of time sufficient to form any RNA cleavage product(s); and (b) detecting any amount of HIV or HCV RNA cleavage product(s) in the sample with a detector. In some cases, the RNA in the sample is not reverse transcribed prior to the detecting step.

In some cases, the methods further comprise a step of amplification of RNA in the sample, or amplification of any HIV or HCV RNA cleavage products that may form. For example, the RNA can be amplified using an RNA-Dependent RNA polymerase or an RNA replicase (EC 2.7.7.48) that can replicate single-stranded RNA. Examples of such RNA replicases include the QB replicase, the RNA Polymerase from Rabbit Hemorrhagic Disease Virus (PDB: 1KHV); the RNA Polymerase from Sapporo Virus (PDB: 2CKW); the Hepatitis C RNA Polymerase (PDB: 2D41); the Neurospora Crassa RNA Polymerase (PDB: 2J7N); the RNA Polymerase Birnavirus (PDB: 2PGG); the RNA Polymerase from Infectious Bursal Disease Virus (PDB: 2PUS); the RNA Polymerase from Rotavirus (PDB: 2R7T); the RNA Polymerase from Infectious Pancreatic Necrosis Virus (PDB: 2Y18); the RNA Polymerase from Cypoviruses (PDB: 3JA4); the Enterovirus A RNA Polymerase

(PDB: 3N6L); the RNA Polymerase from Norwalk Virus (PDB: 3UQS); the RNA Polymerase from Rotavirus A (PDB: 4AU6); the RNA Polymerase from Thosea Assigns Virus (PDB: 4XHA); the Rhinovirus A RNA polymerase (PDB: 1XR7); the Enterovirus C RNA polymerase (PDB: 30L6); the Foot-and-Mouth Disease Virus RNA polymerase (PDB: 1U09); the Cardiovirus A RNA polymerase (PDB: 4NZ0); the Japanese Encephalitis Virus RNA polymerase (PDB: 4HDH); the Bovine Viral Diarrhea Virus 1 RNA polymerase (PDB: 1S48); the Qbeta Virus RNA polymerase (PDB: 3MMP); the Reovirus RNA polymerase (PDB: IMUK); and the La Crosse Bunyavirus RNA polymerase.

In other cases, the RNA and/or the HIV or HCV RNA cleavage product(s) are not amplified.

In some embodiments, the detector is a fluorescence detector, optionally a short quenched-fluorescent RNA detector.

In some embodiments, the at least one HIV crRNA has a sequence such as any one of SEQ ID NO: 1-8. In some embodiments, the sample is incubated with at least two, or at least three, or at least four, or at least five, or at least six, or at least seven, or at least eight, or at least nine, or at least nine, or at least ten, or more crRNAs.

In some embodiments, the HIV or HCV RNA cleavage product concentration is determined using a standard curve. In some embodiments, the HIV or HCV RNA cleavage product concentration is determined using a standard curve and comparing the detected level of the HIV or HCV RNA cleavage against the standard curve.

In some embodiments, the methods further comprise depleting a portion of the sample prior to other step(s) or inhibiting a nuclease in the sample prior to the other step(s). For example, the sample can be depleted of protein, enzymes, lipids, nucleic acids, or a combination thereof. In some embodiments, the depleted portion of the sample is a human nucleic acid portion.

In some embodiments, the methods further comprise removing ribonuclease(s) (RNase) from the sample. In some embodiments, the RNase is removed from the sample using an RNase inhibitor and/or heat.

In some embodiments, the Cas13a protein and/or the crRNA is lyophilized prior to incubation with the sample.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. Furthermore, components can be shown as transparent in certain views for clarity of illustration only and not to indicate that the illustrated component is necessarily transparent.

FIG. 1A demonstrates HIV CRISPR RNA (crRNA) can be used to detect corresponding HXB2 sequences.

FIG. 1B demonstrates HIV crRNAs can be used to detect corresponding HXB2 targets over time.

FIG. 2 demonstrates reactivation of multiple HIV cell lines using RNA isolated from J-Lat cells activated with TNF-α detected by qPCR.

FIGS. 3A-3D demonstrate that not all crRNAs are equally effective.

FIG. 4 demonstrates that not all crRNAs are equally effective overtime.

FIG. 5 is a schematic of the HIV-1 RNA genome and the extensive secondary structure composed of many local and long-distance interactions. Also shown are different crRNA recognition sites within the HIV-1 RNA genome.

FIG. 6 shows specific detection of HIV-1 RNA with Cas13a:crRNA complexes and RNaseAlert detection reagent. Three different crRNAs were tested in total RNAs isolated from J-Lat 11.1 cells reactivated with TNFα (10 ng/ml). Shown are means±SD of three replicate measurements of one RNA sample. Fluorescence values are x10³.

FIG. 7 demonstrates that supernatant (or virion) RNA is more easily detected with lower RNA input compared to cell-associated RNA from two replication competent cell lines.

FIG. 8 demonstrates that the use of multiple crRNAs (multiplexing) can improve detection of HIV-1 RNA.

FIG. 9 demonstrates that Cas13a:crRNA RNP complexes can detect target RNA down to 500 attomole (aM), or 3.01 ×10⁵ copies/mL.

FIG. 10 illustrates that linear regression of a plot of the initial rates of fluorescence (x-intercept in FIG. 9) versus the concentration calculated for each “target” RNA allows a best fit line to be calculated, which provides a standard curve to interpolate concentrations of unknown samples.

FIG. 11 illustrates detection of target HIV using specific combinations of crRNAs. As shown, the combination of crRNAs used are significant in terms of maximally improving sensitivity of the assay. Here, combining RNPs using crRNA #3 and crRNA #5 or crRNA #3 and crRNA #6 give improved results relative to single crRNAs. Combining crRNA #5 and crRNA #6 RNPs does not lead to improved sensitivity compared to using RNPs containing crRNA #5 alone.

FIG. 12 illustrates that isolated HIV RNA can first be amplified using the bacteriophage-derived RNA-dependent RNA polymerase, Qβ replicase. HIV RNA from Vsvg-pseudotyped virions containing HIV NL4-3 with GFP in place of env was incubated with NTPs and with or without Qβ replicase in reaction buffer (100 mM HEPES-NaOH, pH 7.5; 10 mM MgCl₂, and 1 mM EDTA). Amplified RNA was in some cases purified using phenol and subsequently added to the HIV-Cas13a assay. However, in some instances, specified as “no cleanup,” the amplification mixture was directly added to the HIV-Cas13a assay. Legend values reflect input concentrations in the initial amplification reaction.

FIG. 13 shows that cell-associated supernatant HCV RNA was detected robustly with both HCV-specific crRNAs (SEQ ID NO:21 or 25), compared to no detection with mock-treated cells (not shown).

### DETAILED DESCRIPTION

While the present disclosure is capable of being embodied in various forms, the description below of several embodiments is made with the understanding that the present disclosure is to be considered as an exemplification of the invention and is not intended to limit the invention to the specific embodiments illustrated. Headings are provided for convenience only and are not to be construed to limit the invention in any manner. Embodiments illustrated under any heading may be combined with embodiments illustrated under any other heading.

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about.” It also is to be understood, although not always explicitly stated, that the reagents described herein are merely exemplary and that in some cases equivalents may be available in the art.

It must be noted that as used herein and in the appended claims, the singular forms “a”, “an”, and “the” include plural referents unless the context clearly dictates otherwise. Thus, for example, reference to “a cell” includes a plurality of cells.

The term “about” when used before a numerical designation, e.g., temperature, time, amount, concentration, and such other, including a range, indicates approximations which may vary by (+) or (−) 20%, 10%, 5% or 1%.

Also, as used herein, “and/or” refers to and encompasses any and all possible combinations of one or more of the associated listed items, as well as the lack of combinations when interpreted in the alternative (“or”).

The term “treatment” or “treating” in relation to a given disease or disorder, includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving, preventing, or treating symptoms of the disease or disorder. The term “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development or further disease/disorder progression if already present.

The present disclosure provides methods and compositions for diagnosing HIV or HCV infections, quantifying HIV or HCV RNA concentrations, identifying the presence of different HIV or HCV splice variants and/or mutations, and/or monitoring reactivation of HIV or HCV transcription.

Provided herein are methods and compositions for diagnosing HIV or HCV infection comprising incubating a mixture comprising a sample containing RNA, a Cas13a protein, and at least one CRISPR RNA (crRNA) for a period of time to form RNA cleavage products that may be present in the mixture and detecting a level of any such HIV or HCV RNA cleavage products with a detector.

Provided herein are methods and compositions for quantifying HIV or HCV RNA concentration comprising incubating a mixture comprising a sample, a Cas13a protein, and at least one CRISPR guide RNA (crRNA) for a period of time and analyzing the mixture with a detector to determine the concentration of any HIV or HCV RNA(s) that may be present in the mixture.

Provided herein are methods and compositions for identifying the presence or absence of different HIV or HCV splice variants and/or HIV or HCV mutations comprising incubating a mixture comprising a sample containing RNA, a Cas13a protein, and at least one CRISPR guide RNA (crRNA) for a period of time to form any RNA cleavage product(s) that may be present in the mixture, and detecting any different HIV or HCV splice variants and/or any different HIV or HCV mutations by analyzing any RNA cleavage product(s) with a detector, wherein the at least one crRNA recognizes the HIV or HCV splice variants and/or mutations.

Also provided herein are methods and compositions for monitoring reactivation of HIV or HCV transcription comprising incubating a mixture comprising a sample, a Cas13a protein, and at least one CRISPR guide RNA (crRNA) for a period of time and analyzing the mixture for the presence and/or amount of the RNA cleavage product with a detector.

In some aspects provided herein are methods for diagnosing the presence or absence of an HIV or HCV infection comprising incubating a mixture comprising a sample containing RNA, a Cas13a protein, and at least one CRISPR guide RNA (crRNA) for a period of time to form any RNA cleavage product(s) that may be present in the mixture; and detecting level(s) of HIV or HCV RNA cleavage product(s) that may be present in the mixture with a detector, wherein the RNA is not reverse transcribed prior to the detecting step. The presence or absence of an HIV or HCV infection in patient is detected by detecting level of HIV or HCV RNA cleavage product(s) that may be present in the mixture.

In some embodiments, the sample is isolated from a patient. Non-limiting examples of suitable samples include blood, serum, plasma, urine, aspirate, and biopsy samples. Thus, the term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, washed, or enrichment for certain cell populations. The definition also includes sample that have been enriched for particular types of molecules, e.g., RNAs. The term “sample” encompasses biological samples such as a clinical sample such as blood, plasma, serum, aspirate, cerebral spinal fluid (CSF), and also includes tissue obtained by surgical resection, tissue obtained by biopsy, cells in culture, cell supernatants, cell lysates, tissue samples, organs, bone marrow, and the like. A “biological sample” includes biological fluids derived therefrom (e.g., infected cell, etc.), e.g., a sample comprising RNAs that is obtained from such cells (e.g., a cell lysate or other cell extract comprising RNAs). A sample can comprise, or can be obtained from, any of a variety of cells, tissues, organs, or acellular fluids.

In some embodiments, the sample is isolated from a patient known to have or suspected to have HIV. In other embodiments, the sample is isolated from a patient known to not have or suspected to not have HIV. In some embodiments, the HIV is HIV-1, including, for example, HIV-1 group M and HIV-1 group O. In other embodiments, the HIV is HIV-2.

In some embodiments, the sample is incubated with a Cas13a protein (previously known as C2c2). Cas13a binds and cleaves RNA substrates, rather than DNA substrates, which Cas9 can bind. Cas13a contains two HEPN domains for RNA cleavage, consistent with known roles for HEPN domains in other proteins. In some embodiments, the Cas13a proteins are from the following bacteria and/or have at least 75%, at least 80%, at least 85%, at least 90%, at least 95%, at least 98%, at least 99%, or 100% sequence identity to Cas13 in the following bacteria: Leptotrichia wadei, Rhodobacter capsulatus, Herbinix hemicellulosilytica, Leptotrichia buccalis (Lbu), Listeria seeligeri, Paludibacter propionicigenes, Lachnospiraceae bacterium, [Eubacterium] rectale. Listeria newyorkensis, Clostridium aminophilum, and/or Leptotrichia shahii.

For example, a Leptotrichia wadei Cas13a endonuclease can be used that has the following sequence (SEQ ID NO:10; National Center for Biotechnology Information (NCBI) accession no. WP_036059678.1).

| | |
|---|---|
| 1 | mkitkidgvs hykkqdkgil kkkwkdlder kqrekieary |
| 41 | nkqieskiyk effrlknkkr iekeedqnik slyffikely |
| 81 | lnekneewel kninleildd kervikgykf kedvyffkeg |
| 121 | ykeyylrilf nnliekvqne nrekvrknke fldlkeifkk |
| 161 | yknrkidlll ksinnnkinl eykkenvnee iygtnptndr |
| 201 | emtfyellke iiekkdeqks ileekldnfd itnfleniek |
| 241 | ifneeteini ikgkvlnelr eyikekeenn sdnklkqiyn |
| 281 | lelkkyienn fsykkqksks kngkndylyl nflkkimfie |
| 321 | evdekkeink ekfknkinsn fknlfvqkil dygkllyyke |
| 361 | ndeyikntgq letkdleyik tketlirkma vlvsfaansy |
| 401 | ynlfgrvsgd ilgtevvkss ktnvikvgsh ifkekmlnyf |
| 441 | fdfeifdank iveilesisy siynvrngvg hfnklilgky |
| 481 | kkkdintnkr ieedlnnnee ikgyfikkrg eierkvkekf |
| 521 | lsnnlqyyys kekienyfev yefeilkrki pfapnfkrii |
| 561 | kkgedlfnnk nnkkyeyfkn fdknsaeekk eflktrnfll |
| 601 | kelyynnfyk eflskkeefe kivlevkeek ksrgninnkk |
| 641 | sgvsfqsidd ydtkinisdy iasihkkeme rvekyneekq |
| 681 | kdtakyirdf veeifltgfi nylekdkrlh flkeefsilc |
| 721 | nnnnnvvdfn inineekike flkendsktl nlylffnmid |
| 761 | skrisefrne lvkykqftkk rldeekeflg ikielyetli |
| 801 | efviltrekl dtkkseeida wlvdklyvkd sneykeyeei |
| 841 | lklfvdekil sskeapyyat dnktpillsn fektrkygtq |
| 881 | sflseiqsny kyskvekeni edynkkeeie qkkksniekl |
| 921 | qdlkvelhkk weqnkiteke iekynnttrk ineynylknk |
| 961 | eelqnvyllh emlsdllarn vaffnkwerd fkfiviaikq |
| 1001 | flrendkekv neflnppdns kgkkvyfsvs kykntvenid |
| 1041 | gihknfmnli flnnkfmnrk idkmncaiwv yfrnyiahfl |
| 1081 | hlhtknekis lxsqmnllik lfsydkkvqn hilkstktll |
| 1121 | ekyniqinfe isndknevfk ykiknrlysk kgkmlgknnk |
| 1161 | LENEFLE NVKAMLEYSE |

Other sequences for Leptotrichia wadei Cas13a endonucicases are also available, such as those NCBI accession nos. BBM46759.1, BBM48616.1, BBM48974.1, BBM48975.1, and WP_021746003.1.

In another example, a Herbinix hemicellulosilytica Cas13a endonuclease can be used that has the following sequence (SEQ ID NO:11; NCBI accession no. WP_103203632.1).

| | |
|---|---|
| 1 | MKLTRRRISG NSVDQKITAA FYRDMSQGLL YYDSEDNDCT |
| 41 | DKVIESMDFE RSWRGRILKN GEDDKNPFYM FVKGLVGSND |
| 81 | KIVCEPIDVD SDPDNLDILI NKNLTGFGRN LKAPDSNDTL |
| 121 | ENLIRKIQAG IPEEEVLPEL KKIKEMIQKD IVNRKEQLLK |
| 161 | SIKNNRIPFS LEGSKLVPST KKMKWLFKLI DVPNKTFNEK |
| 201 | MLEKYWEIYD YDKLKANITN RLDKTDKKAR SISRAVSEEL |
| 241 | REYHKNLRTN YNRFVSGDRP AAGLDNGGSA KYNPDKEEFL |
| 281 | LFLKEVEQYF KKYFPVKSKH SNKSKDKSLV DKYKNYCSYK |
| 321 | VVKKEVNRSI INQLVAGLIQ QGKLLYYFYY NDTWQEDFLN |
| 361 | SYGLSYIQVE EAFKKSVMTS LSWGINRLTS FFIDDSNTVK |
| 401 | FDDITTKKAK EAIESNYFNK LRTCSRMQDH FKEKLAFFYP |
| 441 | VYVKDKKDRP DDDIENLIVL VKNAIESVSY LRNRTFHFKE |
| 481 | SSLLELLKEL DDKNSGQNKI DYSVAAEFIK RDIENLYDVF |
| 521 | REQIRSLGIA EYYKADMISD CFKTCGLEFA LYSPKNSLMP |
| 561 | AFKNVYKRGA NLNKAYIRDK GPKETGDQGQ NSYKALEEYR |
| 601 | ELTWYIEVKN NDQSYNAYKN LLQLIYYKAF LPEVRENEAL |
| 641 | ITDFINRTKE WNRKETEERL NTKNNKKHKN FDENDDITVN |
| 681 | TYRYESIPDY QGESLDDYLK VLQRKQMARA KEVNEKEEGN |
| 721 | NNYIQFIRDV VVWAFGAYLE NKLKNYKNEL QPPLSKENIG |
| 761 | LNDTLKELFP EEKVKSPFNI KCRFSISTFI DNKGKSTDNT |
| 801 | SAEAVKTDGK EDEKDKKNIK RKDLLCFYLF LRLLDENEIC |
| 841 | KLQHQFIKYR CSLKERRFPG NRTKLEKETE LLAELEELME |
| 881 | LVRFTMPSIP EISAKAESGY DTMIKKYFKD FIEKKVFKNP |
| 921 | KTSNLYYHSD 5KTPVTRKYM ALLMRSAPLH LYKDIFKGYY |
| 961 | LITKKECLEY IKLSNIIKDY QNSLNELHEQ LERIKLKSEK |
| 1001 | QNGKDSLYLD KKDFYKVKEY VENLEQVARY KHLQHKINFE |
| 1041 | SLYRIFRIHV DIAARMVGYT QDWERDMHFL FKALVYNGVL |
| 1081 | EERRFEAIFN NNDDNNDGRI VKKIQNNLNN KNRELVSMLC |
| 1121 | WNKKLNKNEF GAIIWKRNPI AHLNHFTQTE QNSKSSLESL |
| 1161 | INSLRILLAY DRKRQNAVTK TINDLLLNDY HIRIKWEGRV |
| 1201 | DEGQIYFNIK EKEDIENEPI IHLKHLHKKD CYIYKNSYMF |
| 1241 | DKQKEWICNG IKEEVYDKSI LKCIGNLFKF DYEDKNKSSA |
| 1281 | NPKHT |

For example, a Leptotrichia buccalis Cas13a endonuclease can be used that has the following sequence (SEQ ID NO:12: NCBI accession no. WP_015770004.1).

| | |
|---|---|
| 1 | MKVTKVGGIS HKKYTSEGRL VKSESEENRT DERLSALLNM |
| 41 | RLDMYIKNPS STETKENQKR IGKLKKFFSN KMVYLKDNTL |
| 81 | SLKNGKKENI DREYSETDIL ESDVRDKKNF AVLKKIYLNE |
| 121 | NVNSEELEVF RNDIKKKLNK INSLKYSFEK NKANYQKINE |
| 161 | NNIEKVEGKS KRNIIYDYYR ESAKRDAYVS NVKEAFDKLY |
| 201 | KEEDIAKLVL EIENLTKLEK YKIREFYHEI IGRKNDKENF |
| 241 | AKIIYEEIQN VNNMKELIEK VPDMSELKKS QVFYKYYLDK |
| 281 | EELNDKNIKY AFCHFVEIEM SQLLKNYVYK RLSNISNDKI |
| 321 | KRIFEYQNLK KLIENKLLNK LDTYVRNCGK YNYYLQDGEI |
| 361 | ATSDFIARNR QNEAFLRNII GV5SVAYFSL RNILETENEN |
| 401 | DITGRMRGKT VKNNKGEEKY VSGEVDKIYN ENKKNEVKEN |
| 441 | LKMFYSYDFN MDNKNEIEDF FANIDEAISS IRHGIVHFNL |
| 481 | ELEGKDIFAF KNIAPSEISK KMFQNEINEK KLKLKIFRQL |
| 521 | NSANVFRYLE KYKILNYLKR TRFEFVNKNI PFVPSFTKLY |
| 561 | SRIDDLKNSL GIYWKTPKTN DDNKTKEIID AQIYLLKNIY |
| 601 | YGEFLMYFMS NNGNFFEISK EIIELNKNDK RNLKTGFYKL |
| 641 | QKFEDIQEKI PKEYLANIQS LYMINAGNQD EEEKDTYIDF |
| 681 | IQKIFLKGFM TYLANNGRLS LIYIGSDEET NTSLAEKKQE |
| 721 | FDKFLKKYEQ NNNIKIPYEI NEFLREIKLG NILKYTERLN |
| 761 | MFYLILKLLN HKELTNLKGS LEKYQSANKE EAFSDQLELI |
| 801 | NLLNLDNNRV TEDFELEADE IGKFLDFNGN KVKDNKELKK |
| 841 | FDTMKIYFDG ENIIKHRAFY NIKKYGMLNL LEKIADKAGY |
| 881 | KISIEELKKY SNKKNEIEKN HKMQENLHRK YARPRKDEKF |
| 921 | TDEDYESYKQ AIENIEEYTH LKNKVEFNEL NLLQGLLLRI |
| 961 | LHRLVGYTSI WERDLRFRLK GEFPENQYIE EIFNFENKKN |
| 1001 | VKYKGGQIVE KYIKFYKELH QNDEVKINKY SSANIKVLKQ |
| 1041 | EKKDLYIRNY IAHFNYIPHA EISLLEVLEN LRKLLSYDRK |
| 1081 | LKNAVMKSVV DILKEYGFVA TFKIGADKKI GIQTLESEKI |
| 1121 | VHLKNLKKKK LMTDRNSEEL CKLVKIMFEY KMEEKKSEN |

For example, a Leptotrichia seeligeri Cas13a endonuclease can be used that has the following sequence (SEQ ID NO:13: NCBI accession no. WP_012985477.1).

| | |
|---|---|
| 1 | MWISIKTLIH HLGVLFFCDY MYNRREKKII EVKTMRITKV |
| 41 | EVDRKKVLIS RDKNGGKLVY ENEMQDNTEQ IMHHKKSSFY |
| 81 | KSVVNKTICR PEQKQMKKLV HGLLQENSQE KIKVSDVTKL |
| 121 | NISNFLNHRF KKSLYYFPEN SPDKSEEYRI EINLSQLLED |
| 161 | SLKKQQGTFI CWESFSKDME LYIMWAENYI SSKTKLIKKS |
| 201 | IRNNRIQSTE SRSGQLMDRY MKDILNKNKP FDIQSVSEKY |
| 241 | QLEKLTSALK ATFKEAKKND KEINYKLKST LQNHERQIIE |
| 281 | ELKENSELNQ FNIEIRKHLE TYFPIKKTNR KVGDIRNLEI |
| 321 | GEIQKIVNHR LKNKIVQRIL QEGKLASYEI ESTVNSNSLQ |
| 361 | KIKIEEAFAL KFINACLFAS NNLRNMVYPV CKKDILMIGE |
| 401 | FKNSFKEIKH KKFIRQWSQF FSQEITVDDI ELASWGLRGA |
| 441 | IAPIRNEIIH LKKHSWKKFF NNPTFKVKKS KIINGKTKDV |
| 481 | TSEFLYKETL FKDYFYSELD SVPELIINKM ESSKILDYYS |
| 521 | SDQLNQVFTI PNFELSLLTS AVPFAPSFKR VYLKGFDYQN |
| 561 | QDEAQPDYNL KLNIYNEKAF NSEAFQAQYS LFKMVYYQVF |
| 601 | LPQFTTNNDL FKSSVDFILT LNKERKGYAK AFQDIRKMKK |
| 641 | DEKPSEYMSY IQSQLMLYQK KQEEKEKINH FEKFINQVFI |
| 681 | KGFNSFIEKN RLTYICHPTK NTVPENDNIE IPFHTDMDDS |
| 721 | NIAFWLMCKL LDAKQLSELR NEMIKFSCSL QSTEEISTFT |
| 761 | KAREVIGLAL LNGEKGCNDW KELFDDKEAW KKNMSLYVSE |
| 801 | ELLQSLPYTQ EDGQTPVINR SIDLVKKYGT ETILEKLFSS |
| 841 | SDDYKVSAKD IAKLHEYDVT EKIAQQESLH KQWIEKPGLA |
| 881 | RDSAWTKKYQ NVINDISNYQ WAKTKVELTQ VRHLHQLTID |
| 921 | LLSRLAGYMS IADRDFQFSS NYILERENSE YRVTSWILLS |
| 961 | ENKNKNKYND YELYNLKNAS IKVSSKNDPQ LKVDLKQLRL |
| 1001 | TLEYLELFDN RLKEKRNNIS HFNYLNGQLG NSILELFDDA |
| 1041 | RDVLSYDRKL KNAVSKSLKE ILSSHGMEVT FKPLYQTNHH |
| 1081 | LKIDKLQPKK IHHLGEKSTV SSNQVSNEYC QLVRTLLTMK |

For example, a Paludibacter propionicigenes Cas13a endonuclease can be used that has the following sequence (SEQ ID NO:14, NCBI accession no. WP_013443710.1).

| | |
|---|---|
| 1 | MRVSKVKVKD GGKDKMVLVH RKTTGAQLVY SGQPVSNETS |
| 41 | NILPEKKRQS FDLSTLNKTI IKFDTAKKQK LNVDQYKIVE |
| 81 | KIFKYPKQEL PKQIKAEEIL PFLNHKFQEP VKYWKNGKEE |
| 121 | SFNLTLLIVE AVQAQDKRKL QPYYDWKTWY IQTKSDLLKK |
| 161 | SIENNRIDLT ENLSKRKKAL LAWETEFTAS GSIDLTHYHK |
| 201 | VYMTDVLCKM LQDVKPLTDD KGKINTNAYH RGLKKALQNH |
| 241 | QPAIFGTREV PNEANRADNQ LSIYHLEVVK YLEHYFPIKT |
| 281 | SKRRNTADDI AHYLKAQTLK TTIEKQLVNA IRANIIQQGK |
| 321 | TNHHELKADT TSNDLIRIKT NEAFVLNLTG TCAFAANNIR |
| 361 | NMVDNEQTND ILGKGDFIKS LLKDNTNSQL YSFFFGEGLS |
| 401 | TNKAEKETQL WGIRGAVQQI RNNVNHYKKD ALKTVFNISN |
| 441 | FENPTITDPK QQTNYADTIY KARFINELEK IPEAFAQQLK |
| 481 | TGGAVSYYTI ENLKSLLTTF QFSLCRSTIP FAPGFKKVFN |
| 521 | GGINYQNAKQ DESFYELMLE QYLRKENFAE ESYNARYFML |
| 561 | KLIYNNLFLP GFTTDRKAFA DSVGFVQMQN KKQAEKVNPR |
| 601 | KKEAYAFEAV RPMTAADSIA DYMAYVQSEL MQEQNKKEEK |
| 641 | VAEETRINFE KFVLQVFIKG FDSFLRAKEF DFVQMPQPQL |
| 681 | TATASNQQKA DKLNQLEASI TADCKLTPQY AKADDATHIA |
| 721 | FYVFCKLLDA AHLSNLRNEL IKFRESVNEF KFHHLLEIIE |
| 761 | ICLLSADVVP TDYRDLYSSE ADCLARLRPF IEQGADITNW |
| 801 | SDLFVQSDKH SPVIHANIEL SVKYGTTKLL EQIINKDTQF |
| 841 | KTTEANFTAW NTAQKSIEQL IKQREDHHEQ WVKAKNADDK |
| 881 | EKQERKREKS NFAQKFIEKH GDDYLDICDY INTYNWLDNK |
| 921 | MHFVHLMRLH GLTIELLGRM AGFVALFDRD FQFFDEQQIA |
| 961 | DEFKLHGFVN LHSIDKKLNE VPTKKIKEIY DIRNKIIQIN |
| 1001 | GNKINESVRA NLIQFISSKR NYYNNAFLHV SNDEIKEKQM |
| 1041 | YDIRNHIAHF NYLTKDAADF SLIDLINELR ELLHYDRKLK |
| 1081 | NAVSKAFIDL FDKHGMILKL KLNADHKLKV ESIEPKKIYH |
| 1121 | LGSSAKDKPE YQYCTNQVMM AYCNMCRSLL EMKK |

For example, a Lochnospiraceae bacterium Cas13a endonuclease can be used that has the following sequence (SEQ ID NO:15; NCBI accession no. WP_022785443.1).

| | |
|---|---|
| 1 | MKISKVREEN RGAKLTVNAK TAVVSENRSQ EGILYNDPSR |
| 41 | YGKSRKNDED RDRYIESRLK SSGKLYRIFN EDKNKRETDE |
| 81 | LQWFLSEIVK KINRRNGLVL SDMLSVDDRA FEKAFEKYAE |
| 121 | LSYTNRRNKV SGSPAFETCG VDAATAERLK GIISETNFIN |
| 161 | RIKNNIDNKV SEDIIDRIIA KYLKKSLCRE RVKRGLKKLL |
| 201 | MNAFDLPYSD PDIDVQRDFI DYVLEDFYHV RAKSQVSRSI |
| 241 | KNMNMPVQPE GDGKFAITVS KGGTESGNKR SAEKEAFKKF |
| 281 | LSDYAELDER VRDDMLRRMR RLVVLYFYGS DDSKLSDVNE |
| 321 | KFDVWEDHAA RRVDNREFIK LPLENKIANG KTDKDAERIR |
| 361 | KNTVKELYRN QNIGCYRQAV KAVEEDNNGR YFDDKMLNMF |
| 401 | FIHRIEYGVE KIYANLKQVT EFKARTGYLS EKIWKDLINY |
| 441 | ISIKYIAMGK AVYNYAMDEL NASDKKEIEL GKISEEYLSG |
| 481 | ISSFDYELIK AEEMLQRETA VYVAFAARHL SSQTVELDSE |
| 521 | NSDFILLKPK GTMDKNDKNX LASNNILNFL KDKETLRDTI |
| 561 | LQYFGGHSLW TDFPFDKYLA GGKDDVDFLT DLKDVIYSMR |
| 601 | NDSFHYATEN HNNGKWNKEL ISAMFEHETE RMTVVMKDKF |
| 641 | YSNNLPMFYK NDDLKKLLID LYKDNVERAS QVPSFNKVFV |
| 681 | RKNFPALVRD KDNLGIELDL KADADKGENE LKFYNALYYM |
| 721 | FKEIYYNAFL NDKNVRERFI TKATKVADNY DRNKERNLKD |
| 761 | RIKSAGSDEK KKLREQLQNY IAENDFGQRI KNIVQVNPDY |
| 801 | TLAQICQLIM TEYNQQNNGC MQKKSAARKD INKDSYQHYK |
| 841 | MLLLVNIRKA FLEFIKENYA FVLKPYKHDL CDKADFVPDF |
| 881 | AKYVKPYAGL ISRVAGSSEL QKWYIVSRFL SPAQANHMLG |
| 921 | FLHSYKQYVW DIYRRASETG TEINHSIAED KIAGVDITDV |
| 961 | DAVIDLSVKL CGTISSEISD YFKDDEVYAE YISSYLDFEY |
| 1001 | DGGNYKDSLN RFCNSDAVND QKVALYYDGE HPKLNRNIIL |
| 1041 | SKLYGERRFL EKITDRVSRS DIVEYYKLKK ETSQYQTKGI |
| 1081 | FDSEDEQKNI KKFQEMKNIV EFRDLMDYSE IADELQGQLI |
| 1121 | NWIYLRERDL MNFQLGYHYA CLNNDSNKQA TYVTLDYQGK |
| 1161 | KNRKINGAIL YQICAMYING LPLYYVDKDS SEWTVSDGKE |
| 1201 | STGAKIGEFY RYAKSFENTS DCYASGLEIF ENISEHDNIT |
| 1241 | ELRNYIEHFR YYSSFDRSFL GIYSEVFDRF FTYDLKYRKN |
| 1281 | VPTILYNILL QHFVNVRFEF VSGKKMIGID KKDRKIAKEK |
| 1321 | ECARITIREK NGVYSEQFTY KLKNGTVYVD ARDKRYLQSI |
| 1361 | IRLLFYPEKV NMDEMIEVKE KKKPSDNNTG KGYSKRDRQQ |
| 1401 | DRKEYDKYKE KKKKEGNFLS GMGGNINWDE INAQLKN |

For example, a Leptotrichia shahii Cas13a endonuclease can be used that has the following sequence (SEQ ID NO:16; NCBI accession no. BBM39911.1).

| | |
|---|---|
| 1 | MGNLFGHKRW YEVRDKKDFK IKRKVKVKRN YDGNKYILNI |
| 41 | NENNNKEKID NNKFIRKYIN YKKNDNILKE FTRKFHAGNI |
| 81 | LFKLKGKEGI IRIENNDDFL ETEEVVLYIE AYGKSEKLKA |
| 121 | LGITKKKIID EAIRQGITKD DKKIEIKRQE NEEEIEIDIR |
| 161 | DEYTNKTLND CSIILRIIEN DELETKKSIY EIFKNINMSL |
| 201 | YKIIEKIIEN ETEKVFENRY YEEHLREKLL KDDKIDVILT |
| 241 | NFMEIREKIK SNLEILGFVK FYLNVGGDKK KSKNKKMLVE |
| 281 | KILNINVDLT VEDIADFVIK ELEFWNITKR IEKVKKVNNE |
| 321 | FLEKRRNRTY IKSYVLLDKH EKFKIFRENK KDKIVKFFVE |
| 361 | NIKNNSIKEK IEKILAEFKI DELIKKLEKE LKKGNCDTEI |
| 401 | FGIFKKHYKV NFDSKKFSKK SDEEKELYKI IYRYLKGRIE |
| 441 | KILVNEQKVR LKKMEKIEIE KILNESILSE KILKRVKQYT |
| 481 | LEHIMYLGKL RHNDIDMTTV NTDDFSRLHA KEELDLELIT |
| 521 | FFASTNMELN KIFSRENINN DENIDFFGGD REKNYVLDKK |
| 561 | ILNSKIKIIR DLDFIDNKNN ITNNFIRKFT KIGTNERNRI |
| 601 | LHAISKERDL QGTQDDYNKV INIIQNLKIS DEEVSKALNL |
| 641 | DVVFKDKKNI ITKINDIKIS EENNNDIKYL PSFSKVLPEI |
| 681 | LNLYRNNPKN EPFDTIETEK IVLNALIYVN KELYKKLILE |
| 721 | DDLEENESKN IFLQELKKTL GNIDEIDENI IENYYKNAQI |
| 761 | SASKGNNKAI KKYQKKVIEC YIGYLRKNYE ELFDFSDFKM |
| 801 | NIQEIKKQIK DINDNKTYER ITVKTSDKTI VINDDFEYII |
| 841 | SIFALLNSNA VINKIRNRFF ATSVWLNTSF YQNIIDILDE |
| 881 | IMQLNTLRNE CITENWNLNL EEFIQKMKEI EKDFDDFKIQ |
| 921 | TKKEIFNNYY EDIKNNILTE FKDDINGCDV LEKKLEKIVI |
| 961 | FDDETKFEID KKSNILQDEQ RKLSNINKKD LKKKVDQYIK |
| 1001 | DKDQEIKSKI LCRIIFNSDF LKKYKKEIDN LIEDMESENE |
| 1041 | NKFQEIYYPK ERKNELYIYK KNLFLNIGNP NFDKIYGLIS |
| 1081 | NDIKMADAKF LFNIDGKNIR KNKISEIDAI LKNLNDKLNG |
| 1121 | YSKEYKEKYI KKLKENDDFF AKNIQNKNYK SFEKDYNRVS |
| 1161 | EYKKIRDLVE FNYLNKIESY LIDINWKLAI QMARFERDMH |
| 1201 | YIVNGLRELG IIKLSGYNTG ISRAYPKRNG SDGFYTTTAY |
| 1241 | YKFFDEESYK KFEKICYGFG IDLSENSEIN KPENESIRNY |
| 1281 | ISHFYIVRNP FADYSIAEQI DRVSNLLSYS TRYNNSTYAS |
| 1321 | VFEVFKKDVN LDYDELKKKF KLIGNNDILE RLMKPKKVSV |
| 1361 | LELESYNSDY IKNLIIELLT KIENTNDTL |

In another example, a Leptotrichia buccalis C-1013-b Cas13a endonuclease can be used that has the following sequence (SEQ ID NO: 17; NCBI accession no. C7NBY4, AltName LbuC2c2).

| | |
|---|---|
| 1 | MKVTKVGGIS HKKYTSEGRL VKSESEENRT DERLSALLNM |
| 41 | RLDMYIKNPS STETKENQKR IGKLKKFFSN KMVYLKDNTL |
| 81 | SLKNGKKENI DREYSETDIL ESDVRDKKNF AVLKKIYLNE |
| 121 | NVNSEELEVF RNDIKKKLNK INSLKYSFEK NKANYQKINE |
| 161 | NNIEKVEGKS KRNIIYDYYR ESAKRDAYVS NVKEAFDKLY |
| 201 | KEEDIAKLVL EIENLTKIEK YKIREFYHEI IGRKNDKENF |
| 241 | AKIIYEEIQN VNNMKELIEK VPDMSELKKS QVFYKYYLDK |
| 281 | EELNDKNIKY AFCHFVEIEM SQLLKNYVYK RLSNISNDKI |
| 321 | KRIFEYQNLK KLIENKLLNK LDTYVRNCGK YNYYLQDGEI |
| 361 | ATSDFIARNR QNEAFLRNII GVSSVAYFSL RNILETENEN |
| 401 | DITGRMRGKT VKNNKGEEKY VSGEVDKIYN ENKKNEVKEN |
| 441 | LKMFYSYDEN MDNKNEIEDF FANIDEAISS IRHGIVHFNL |
| 481 | ELEGKDIFAF KNIAPSEISK KMFQNEINEK KLKLKIFRQL |
| 521 | NSANVFRYLE KYKILNYLKR TRFEFVNKNI PFVPSFTKLY |
| 561 | SRIDDLKNSL GIYWKTPKTN DDNKTKEIID AQIYLLKNIY |
| 601 | YGEFLNYFMS NNGNFFEISK EIIELNKNDK RNLKTGFYKL |
| 641 | QKFEDIQEKI PKEYLANIQS LYMINAGNQD EEEKDTYIDF |
| 681 | IQKIFLKGFM TYLANNGRLS LIYIGSDEET NTSLAEKKQE |
| 721 | FDKFLKKYEQ NNNIKIPYEI NEFLREIKLG NILKYTERLN |
| 761 | MFYLILKLLN HKELTNLKGS LEKYQSANKE EAFSDQLELI |
| 801 | NLLNLDNNRV TEDFELEADE IGKFLDFNGN KVKDNKELKK |
| 841 | FDTNKIYFDG ENIIKHRAFY NIKKYGMLNL LEKIADKAGY |
| 881 | KISIEELKKY SNKKNEIEKN HKMQENLHRK YARPRKDEKF |
| 921 | TDEDYESYKQ AIENIEEYTH LKNKVEFNEL NLLQGLLLRI |
| 961 | LHRLVGYTSI WERDLRFRLK GEFPENQYIE EIFNFENKKN |
| 1001 | VKYKGGQIVE KYIKFYKELH QNDEVKINKY SSANIKVLKQ |
| 1041 | EKKDLYIRNY IAHFNYIPHA EISLLEVLEN LRKLLSYDRK |
| 1081 | LKNAVMKSVV DILKEYGFVA TFKIGADKKI GIQTLESEKI |
| 1121 | VHLKNLKKKK LMTDRNSEEL CKLVKIMFEY KMEEKKSEN |

In some embodiments, the sample is incubated with at least one CRISPR RNA (crRNA) and the Cas13a protein. However, incubation of the crRNA and Cas13a protein without the sample is preferred, so that the crRNA and the Cas13a protein can form a complex. The sample RNA (e.g., HIV or HCV RNA) can then be added and act as an activating RNA. Once activated by complex formation with the activating RNA, the crRNA/Cas13a complex becomes a non-specific RNase to produce RNA cleavage products that can be detected using, for example, a short quenched-fluorescent RNA.

For example, the Cas13a and crRNA are incubated for a period of time to form the inactive complex. In some embodiments, the Cas13a and crRNA complexes are formed by incubating together at 37° C. for 30 minutes, 1 hour, or 2 hours (for example, 0.5 to 2 hours) to form an inactive complex. The inactive complex can then be incubated with the RNase Alert Substrate “Target” and complementary ssRNA activator (e.g., sample HIV or HCV RNA) so that the Cas13a/crRNA becomes an activated complex that cleaves in cis and trans. The at least one crRNA can bind to a region in the HIV RNA genome or in the HCV RNA genome. In some embodiments, the region is a single stranded region of the HIV or HCV RNA genome. In other embodiments, the region is a hairpin region of the HIV RNA or HCV genome.

In some embodiments, the at least one HIV crRNA is any one of SEQ ID NOs: 1-8. In some embodiments, the at least one crRNA has about 70%, about 75%, about 80%, about 85%, about 90%, about 95%, or more sequence identity to any one of SEQ ID NO 1-8. Table 1 provides examples of HIV crRNAs.

TABLE 1
Examples of HIV crRNA Sequences

| SEQ ID NO | Name | Sequence |
|---|---|---|
| SEQ ID NO: 1 | PF001_crLbu_HXB2_1 | GACCACCCCAAAAAUGAAGGGGACUA |
| | | AACUUUUUUUUUUUUUGAAGCAC |
| SEQ ID NO: 2 | PF002_crLbu_HXB2_2 | GACCACCCCAAAAAUGAAGGGGACUA |
| | | AAACCUGCUUAUAUGCAGGAUCUG |
| SEQ ID NO: 3 | PF003_crLbu_HXB2_3 | GACCACCCCAAAAAUGAAGGGGACUA |
| | | AAACCCAGAGAGACCCAGUACAGG |
| SEQ ID NO: 4 | PF004_crLbu_HXB2_4 | GACCACCCCAAAAAUGAAGGGGACUA |
| | | AAACCCUGCCAUAGGAGAUGCCUA |
| SEQ ID NO: 5 | PF005_crLbu_HXB2_5 | GACCACCCCAAAAAUGAAGGGGACUA |
| | | AAACGUCUCCGCUUCUUCCUGCCA |
| SEQ ID NO: 6 | PF006_crLbu_HXB2_6 | GACCACCCCAAAAAUGAAGGGGACUA |
| | | AAACAGCUUGAUGAGUCUGACUGU |
| SEQ ID NO: 7 | PF007_crLbu_HXB2_7 | GACCACCCCAAAAAUGAAGGGGACUA |
| | | AAACUUCCUUCGGGCCUGUCGGGU |
| SEQ ID NO: 8 | PF008_crLbu_HXB2_8 | GACCACCCCAAAAAUGAAGGGGACUA |
| | | AAACGGAUCUGUCUCUGUCUCUCU |
| SEQ ID NO: 9 | Stem for all crRNAs | GACCACCCCAAAAAUGAAGGGGACUA |
| | | AAAC |

In some embodiments, the at least one HCV crRNA is any one of SEQ ID NOs: 18-26. In some embodiments, the at least one crRNA has about 70%, about 75%, about 80%, about 85%, about 90%, about 95%, or more sequence identity to any one of SEQ ID NO: 18-26. Table 2 provides examples of HCV crRNAs.

TABLE 2
Examples of HCV crRNA Sequences

| SEQ | | |
|---|---|---|
| ID | | |
| NO: | Name | Sequence |
| 18 | PF016_crLbu_JFH1_1 | GACCACCCCAAAAAUGAAGGGGACUAA |
| | | AACGUGUACUCACCGGUUCCGCA |
| 19 | PF017_crLbu_JFH1_2 | GACCACCCCAAAAAUGAAGGGGACUAA |
| | | AACCCCUAUCAGGCAGUACCACA |
| 20 | PF018_crLbu_JFH1_3 | GACCACCCCAAAAAUGAAGGGGACUAA |
| | | AACACCGGGUAGGUUCCCUGUUG |
| 21 | PF019_crLbu_JFH1_4 | GACCACCCCAAAAAUGAAGGGGACUAA |
| | | AACGGGCGACCAGUUCAUCAUCA |
| 22 | PF020_crLbu_JFH1_5 | GACCACCCCAAAAAUGAAGGGGACUAA |
| | | AACGACGAUGACCUUCUUCUCCA |
| 23 | PF021_crLbu_JFH1_6 | GACCACCCCAAAAAUGAAGGGGACUAA |
| | | AACUUCCACUGCCAGUUGGAGCA |
| 24 | PF022_crLbu_JFH1_7 | GACCACCCCAAAAAUGAAGGGGACUAA |
| | | AACGUUCAUCCAUUGGACCGCGC |
| 25 | PF023_crLbu_JFH1_8 | GACCACCCCAAAAAUGAAGGGGACUAA |
| | | AACGGCUCGAGAAAGUCCAGAAC |
| 26 | PF024_crLbu_JFH1_9 | GACCACCCCAAAAAUGAAGGGGACUAA |
| | | AACUCUGCAGAGAGACCAGUUAC |

In some embodiments, the at least one crRNA recognizes the HIV or HCV splice variants and/or mutations.

In some embodiments, the sample is incubated with a single crRNA. In other embodiments, the sample is incubated with 2, 3, 4, 5, 6, 7, 8, 9, 10 or more crRNAs having a different sequence.

In some embodiments, the Cas13a protein and/or crRNA is lyophilized prior to incubation with the sample.

In some embodiments, the sample is incubated with the Cas13a protein and crRNA for a period of time sufficient to form RNA cleavage products. In some embodiments, the period of time is about 5 hours or less, about 4 hours or less, about 3 hours or less, about 2 hours or less, about 1.5 hours or less, about 1 hour or less, about 40 minutes or less, about 35 minutes or less, about 30 minutes or less, about 25 minutes or less, about 20 minutes or less, about 15 minutes or less, about 10 minutes or less, about 5 minutes or less, or about 1 minute or less.

In some embodiments, the RNA cleavage products (e.g., HIV or HCV RNA cleavage products) are detected using detector RNA using a fluorescence-emitting dye pair, i.e., a fluorescence resonance energy transfer (FRET) pair and/or a quencher/fluorophore pair.

In some embodiments, HIV or HCV RNA, and/or the RNA cleavage products (e.g., HIV or HCV RNA cleavage products) are present in the sample or the mixture along with non-target RNA (e.g., non-HIV RNA or non-HCV RNA). In some embodiments, the RNA cleavage products (e.g., HIV or HCV RNA cleavage products) are present at from about one copy per 10¹⁰ non-target RNAs (e.g., non-HIV or non-HCV RNAs) to about one copy per 10 non-target RNAs (e.g., non-HIV or non-HCV RNAs), at from about one copy per 10⁹ non-target RNAs (e.g., non-HIV RNA or non-HCV RNAs) to about one copy per 10² non-target RNAs (e.g., non-HIV RNA or non-HCV RNAs), at from about one copy per 10⁸ non-target RNAs (e.g., non-HIV RNA or non-HCV RNAs) to about one copy per 10³ non-target RNAs (e.g., non-HIV RNA or non-HCV RNAs), at from about one copy per 10⁷ non-target RNAs (e.g., non-HIV RNA or non-HCV RNAs) to about one copy per 10⁴ non-target RNAs (e.g., non-HIV RNA or non-HCV RNAs), at from about one copy per 10⁶ non-target RNAs (e.g., non-HIV RNA or non-HCV RNAs) to about one copy per 10⁵ non-target RNAs (e.g., non-HIV RNA or non-HCV RNAs), at from about one copy per 10¹⁰ non-target RNAs (e.g., non-HIV RNAs or non-HCV RNAs) to about one copy per 100 non-target RNAs (e.g., non-HIV RNAs or non-HCV RNAs), at from about one copy per 10⁹ non-target RNAs (e.g., non-HIV RNAs or non-HCV RNAs) to about one copy per 100 non-target RNAs (e.g., non-HIV RNAs or non-HCV RNAs), at from about one copy per 10⁸ non-target RNAs (e.g., non-HIV RNAs or non-HCV RNAs) to about one copy per 100 non-target RNAs (e.g., non-HIV RNAs or non-HCV RNAs), at from about one copy per 10⁷ non-target RNAs (e.g., non-HIV RNA sor non-HCV RNAs) to about one copy per 100 non-target RNAs (e.g., non-HIV RNAs or non-HCV RNAs), at from about one copy per 10⁶ non-target RNAs (e.g., non-HIV RNAs or non-HCV RNAs) to about one copy per 100 non-target RNAs (e.g., non-HIV RNAs or non-HCV RNAs), at from about one copy per 10⁵ non-target RNAs (e.g., non-HIV RNAs or non-HCV RNAs) to about one copy per 100 non-target RNAs (e.g., non-HIV RNAs or non-HCV RNAs), at from about one copy per 10⁴ non-target RNAs (e.g., non-HIV RNAs or non-HCV RNAs) to about one copy per 100 non-target RNAs (e.g., non-HIV RNAs or non-HCV RNAs), or at from about one copy per 10³ non-target RNAs (e.g., non-HIV RNAs or non-HCV RNAs) to about one copy per 100 non-target RNAs (e.g., non-HIV RNA or non-HCV RNAs).

In some embodiments, the methods described and disclosed herein can detect an amount of RNA cleavage products (e.g., HIV or HCV RNA cleavage products) in an amount of about 10 nM or less, about 5 nM or less, about 1 nM or less, about 0.5 nM or less, about 0.1 nM or less, about 0.05 nM or less, about 0.01 nM or less, about 0.005 nM or less, about 0.001 nM or less, about 0.0005 nM or less, about 0.0001 nM or less, about 0.00005 nM or less, or about 0.00001 nM or less. In some embodiments, the methods described and disclosed herein can detect an amount of RNA cleavage products (e.g., HIV or HCV RNA cleavage products) in an amount of about 10 pM or less, about 5 pM or less, about 1 pM or less, about 0.5 pM or less, about 0.1 pM or less, about 0.05 pM or less, about 0.01 pM or less, about 0.005 pM or less, about 0.001 pM or less, about 0.0005 pM or less, about 0.0001 pM or less, about 0.00005 pM or less, or about 0.00001 pM or less. In some embodiments, the methods described and disclosed herein can detect an amount of RNA cleavage products (e.g., HIV or HCV RNA cleavage products) in an amount of about 100fM or less, about 50 fM or less, about 25 fM or less, about 20 fM or less, about 15 fM or less, about 10 fM or less, about 5 fM or less, or about 1fM or less.

In some embodiments, the methods described and disclosed herein can detect an amount of RNA cleavage products (e.g., HIV or HCV RNA cleavage products) in an amount of about 1fM or more, about 5 fM or more, about 10 fM or more, about 15 fM or more, about 20 fM or more, about 25 fM or more, about 50 fM or more, about 100 fM or more. In some embodiments, the methods described and disclosed herein can detect an amount of RNA cleavage products (e.g., HIV or HCV RNA cleavage products) in an amount of about 0.00001 pM or more, about 0.00005 pM or more, about 0.0001 pM or more, about 0.0005 pM or more, about 0.001 pM or more, about 0.005 pM or more, about 0.01 pM or more, about 0.05 pM or more, about 0.1 pM or more, about 0.5 pM or more, about 1 pM or more, about 5 pM or more, or about 10 pM or more. In some embodiments, the methods described and disclosed herein can detect an amount of RNA cleavage products (e.g., HIV or HCV RNA cleavage products) in an amount of about 0.00001 nM or more, about 0.00005 nM or more, about 0.0001 nM or more, about 0.0005 nM or more, about 0.001 nM or more, about 0.005 nM or more, about 0.01 nM or more, about 0.05 nM or more, about 0.1 nM or more, about 0.5 nM or more, about 1 nM or more, about 5 nM or more, or about 10 nM or more.

In some embodiments, the methods described and disclosed herein can detect an amount of RNA cleavage products (e.g., HIV or HCV RNA cleavage products) in an amount of from about 10⁶ nM to about 1 nM, from about 10⁶ nM to about 5×10⁶ nM, from about 5×10⁶ nM to about 105 nM, from about 10⁵ nM to about 5×10⁵ nM, from about 5×10⁵ nM to about 10⁴ nM, from about 10⁴ nM to about 5×10⁴ nM, from about 5×10⁴ nM to about 10³ nM, from about 10³ nM to about 5×10³ nM, from about 5×10³ nM to about 10² nM, from about 10² nM to about 5×10² nM, from about 5×10² nM to about 0.1 nM, from about 0.1 nM to about 0.5 nM, from about 0.5 nM to about 1 nM, from about 1 nM to about 5 nM, or from about 5 nM to about 10 nM.

In some embodiments, the methods include detecting a level of the RNA cleavage product (e.g., HIV or HCV RNA cleavage products) with a detector. Detection of the RNA cleavage product can occur by any method known to one of skill in the art. Non-limiting examples of suitable detectors include gold nanoparticle-based detectors, fluorescence polarization, colloid phase transition/dispersion, electrochemical detection, semiconductor-based sensing, and detection of a labeled detector RNA. In some embodiments, the labeled detector is a fluorescence detector, optionally a short quenched-fluorescent RNA. The readout of such detectors can be any convenient readout, including mobile phone-based detectors, to read a measured amount of detectable fluorescent signal; a visual analysis of bands on a gel (e.g., bands that represent cleaved product versus uncleaved substrate), a visual or sensor based detection of the presence or absence of a color (i.e., color detection method), and the presence or absence of (or a particular amount of) an electrical signal.

In some embodiments, the RNA cleavage product concentration is determined using a standard curve and the level of the RNA cleavage product. In some embodiments, a standard curve can be created by running a series of different, known concentrations of “target” ssRNAs with complementarity to the crRNA(s) (that complex with Cas13a protein) and a reporter. Fluorescence can then be tracked over a period of time, for example, over about 10 minutes, over about 20 minutes, over about 30 minutes, over about 45 minutes, over about 1 hour, over about 2 hours, over about 3 hours, over about 4 hours, over about 5 hours, over about 6 hours, or more. In some embodiments, the fluorescence is tracked for over about 2 hours. The initial rate of each reaction is then determined and plotted to create a linear standard curve. In parallel, a sample of unknown concentration is also run. The initial rate of the fluorescence curve (e.g., 2-hour fluorescence curve) for the unknown sample is, for example, plotted on the standard curve to interpolate the concentration of HIV-1 RNA.

In some embodiments, the RNA is not reverse transcribed prior to the detecting step. In some embodiments, the methods further comprise comprising a step of amplification of the RNA and/or the RNA cleavage product. In other embodiments, the methods do not comprise comprising a step of amplification of the RNA and/or the RNA cleavage product. In some embodiments, the methods do not include reverse transcribing the RNA prior to the detecting step and do not amplify the RNA and/or RNA cleavage product.

In some embodiments, a portion of the sample or the reaction mixture is depleted prior to the detecting step. A non-limiting example of a suitable method for depletion is Depletion of Abundant Sequences by Hybridization (DASH) as described in US Publication No. 2018/0051320 which is incorporated by reference in its entirety. In some embodiments, the portion of the sample that is depleted is a human nucleic acid portion, for example human RNA.

In some embodiments, RNase is removed from the sample. In some embodiments, RNase is removed from the sample using an RNase inhibitor and/or heat.

Also described herein are kits that are useful for performing the methods detailed herein. Such kits can include a package that has at least one Cas13a protein, at least one CRISPR guide RNA (crRNA), and instructions for performing a method described herein, where each of the CRISPR guide RNA(s) can have a sequence with about 70%, about 75%, about 80%, about 85%, about 90%, about 95%, or more sequence identity to any one of SEQ ID NO: 1-8, 18-25 or 26. The kit can also contain at least one short quenched-fluorescent RNA.

The CRISPR guide RNAs (crRNAs) can be provided in an array where each crRNA is present within a well of a microarray or where each type of crRNA is attached to a discrete location on a solid surface. The crRNA(s) are supplied in a form that allows or facilitates complex formation with at least one Cas13a protein. Hence, any crRNAs are attached to a solid surface in a manner that does not interfere with complex formation with at least one Cas13a protein.

The kits can also include components such as nuclease-free water, a buffer to regulate the pH of a solution, reaction vessel(s), and/or implements for collection of a sample from a patient. Implements for collection of a sample can include at least one receptacle for a sample, at least one lancet, a capillary tube for collection of small samples (e.g., blood), at least one needle, at least one syringe, at least one alcohol swab, a nuclease inhibitor (e.g., an RNase inhibitor), at least one component for stabilizing the sample, or a combination thereof.

Optionally, the methods include a further step of treating HIV or HCV in subjects where HIV or HCV is detected or where monitored HIV or HCV levels have increased. Such a method can include administration of a therapeutic agent to a patient with detectable HIV or HCV.

Such treatment when HIV is detected can involve antiretroviral therapy (ART), combined antiretroviral therapy (cART), high active antiretroviral therapy (HAART), latency reversal agent(s) (LRAs), integrase strand transfer inhibitor (INSTI)-based regimens, regimens that include one or more boosted protease inhibitor active against HIV-2 (e.g., darunavir or lopinavir), or a combination of HIV therapeutic agents.

Such treatment when HCV is detected can, for example, involve administration of a therapeutic agent such as Daclatasir, Elbasvir-Grazoprevir, Glecaprevir-Pibrentasvir, Harvoni, Ledipasvir-Sofosbuvir, Ombitasvir-Paritaprevir-Ritonavir, Ombitasvir-Paritaprevir-Ritonavir and Dasabuvir, Peginterferon alfa-2a, Peginterferon alfa-2b, Ribavirin, Simeprevir, Sofosbuvir, Technivie, Sofosbuvir-Velpatasvir, Sofosbuvir-Velpatasvir-Voxilaprevir, Zepatier, or combinations thereof.

In some embodiments, the kits described herein can also include a therapeutic agent for treatment of HIV or HCV.

### EXAMPLES

### Example 1: Validation of Cas13a Detection of HIV Transcripts

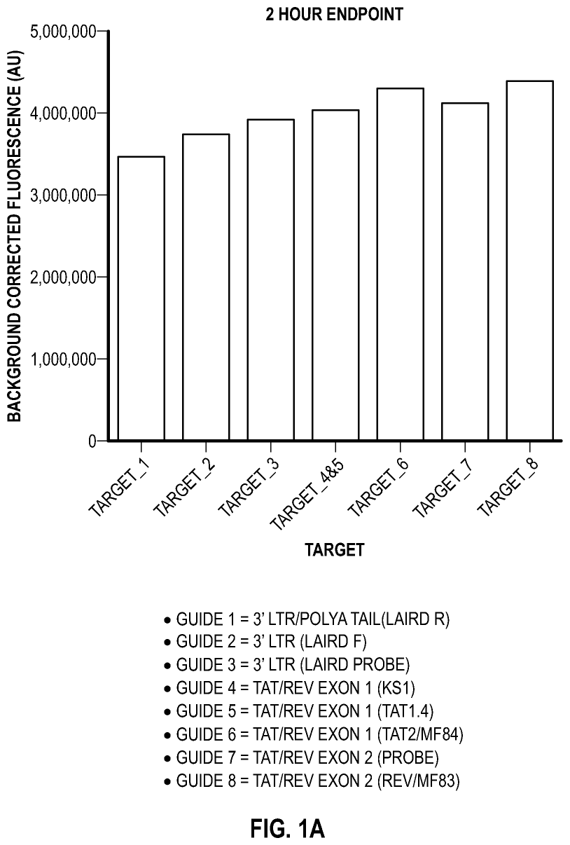

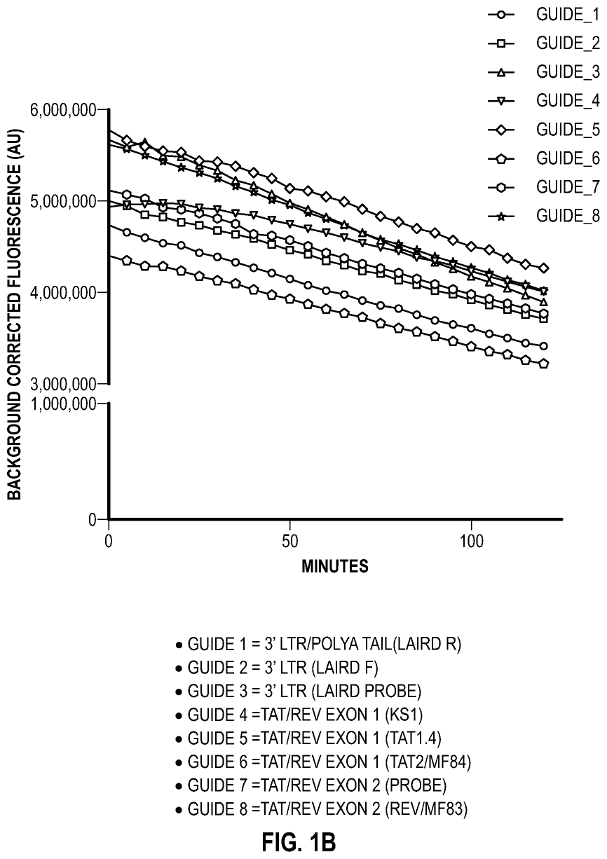

CRISPR RNA guides (crRNAs) were designed and validated for HIV. Eight crRNAs were designed with 20-nt spacers corresponding to TILDA (tat/rev) & Laird et al. (J. Clin Invest 125 (5): 1901-1912 (2015)) (3′LTR/polyA tail). Each crRNA includes a crRNA stem that is derived from a bacterial sequence, while the spacer sequence is derived from the HIV genome (reverse complement). See Table 1 (reproduced below) for crRNA sequences. The crRNAs were validated against 50 nucleotide ssRNAs spanning corresponding portions of the HXB2 genome. Results demonstrated that the HIV crRNAs detected their corresponding HXB2 “activators” and HXB2 targets, as illustrated in FIGS. 1A and 1B.

TABLE 1
Examples of HIV crRNA Sequences

| SEQ ID NO | Name | Sequence |
|---|---|---|
| SEQ ID NO: 1 | PF001_crLbu_HXB2_1 | GACCACCCCAAAAAUGAAGGGGACUA |
| | | AACUUUUUUUUUUUUUGAAGCAC |
| SEQ ID NO: 2 | PF002_crLbu_HXB2_2 | GACCACCCCAAAAAUGAAGGGGACUA |
| | | AAACCUGCUUAUAUGCAGGAUCUG |
| SEQ ID NO: 3 | PF003_crLbu_HXB2_3 | GACCACCCCAAAAAUGAAGGGGACUA |
| | | AAACCCAGAGAGACCCAGUACAGG |
| SEQ ID NO: 4 | PF004_crLbu_HXB2_4 | GACCACCCCAAAAAUGAAGGGGACUA |
| | | AAACCCUGCCAUAGGAGAUGCCUA |
| SEQ ID NO: 5 | PF005_crLbu_HXB2_5 | GACCACCCCAAAAAUGAAGGGGACUA |
| | | AAACGUCUCCGCUUCUUCCUGCCA |
| SEQ ID NO: 6 | PF006_crLbu_HXB2_6 | GACCACCCCAAAAAUGAAGGGGACUA |
| | | AAACAGCUUGAUGAGUCUGACUGU |
| SEQ ID NO: 7 | PF007_crLbu_HXB2_7 | GACCACCCCAAAAAUGAAGGGGACUA |
| | | AAACUUCCUUCGGGCCUGUCGGGU |
| SEQ ID NO: 8 | PF008_crLbu_HXB2_8 | GACCACCCCAAAAAUGAAGGGGACUA |
| | | AAACGGAUCUGUCUCUGUCUCUCU |
| SEQ ID NO: 9 | Stem for all crRNAs | GACCACCCCAAAAAUGAAGGGGACUA |
| | | AAAC |

The crRNAs were then tested using several HIV cell lines. Briefly, crRNAs were diluted to 28 μM in TE buffer, pH 8. Activator ssRNA targets were prepared at 100 μM in TE buffer, pH 8. In some experiments, RNA from J-Lat cells (e.g., NIH AIDS Reagent Program catalog #9849) was used as the activator ssRNA target. J-Lat cells are Jurkat-derived human T cells that are latently infected with the packaged retroviral construct HIV-R7/E-/GFP, which is full length HIV-1 minus env, minus nef. J-Lat RNA was calculated to 325 ng in 0.65 μL water containing diethyl pyrocarbonate (DEPC water). A complex master mix was made by mixing 5× buffer, DEPC water, and crRNA, which was then combined with Cas13a protein. Controls without Cas13a protein were also made. The complex master mixes were then incubated at 37° C. for 30-60 minutes. DEPC water was added to lyophilized RNaseAlert (ThermoFisher Scientific) to resuspend. Target mixes were then made by adding RNase Alert, DEPC water, and 5× buffer to individually aliquoted HIV RNA activator samples. Five uL of target mix was added to each well of a 384-well plate along with 15 pL of appropriate complex master mix. The formation of RNA cleavage products was monitored with a fluorometer.

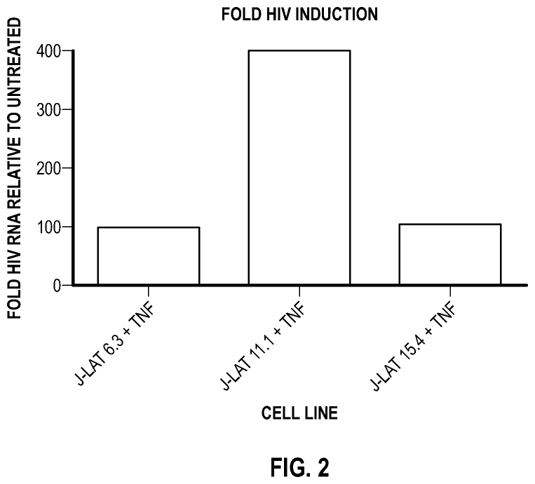

Multiple HIV cell lines were reactivated with tumor necrosis factor alpha (TNF-α). FIG. 2 illustrate the amount of reactivated HIV detected by determining the amount of HIV RNA in the different reaction mixtures.

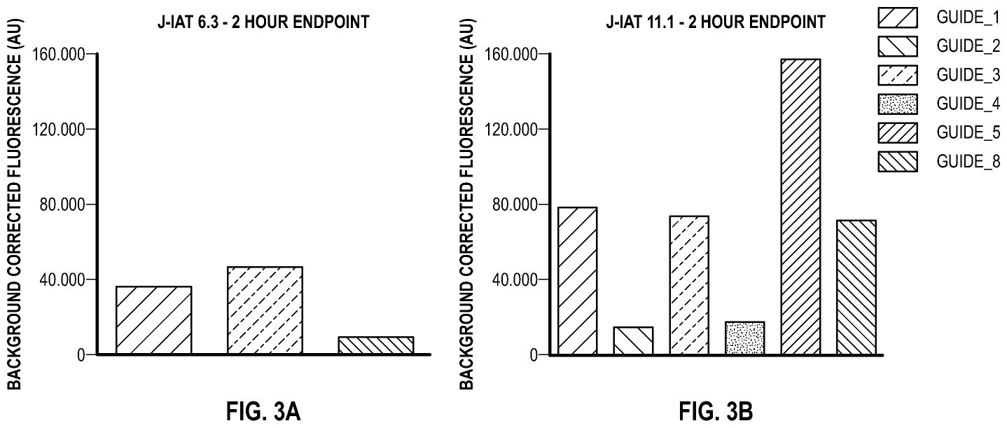

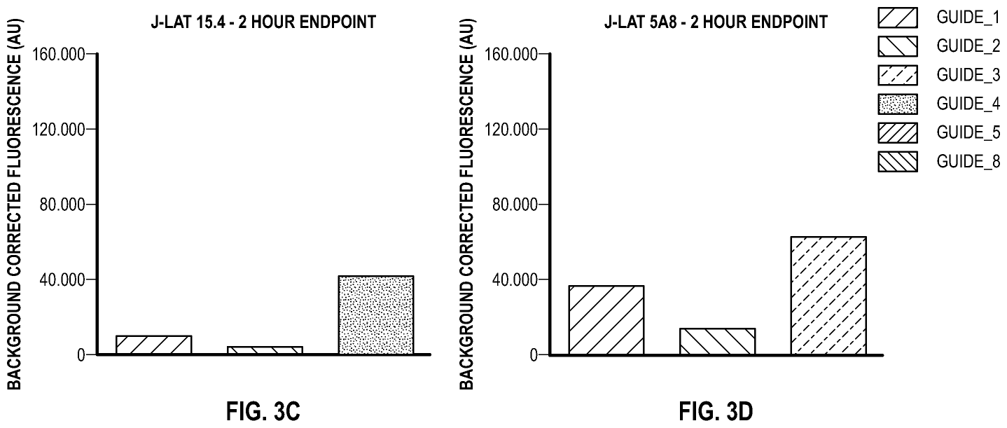

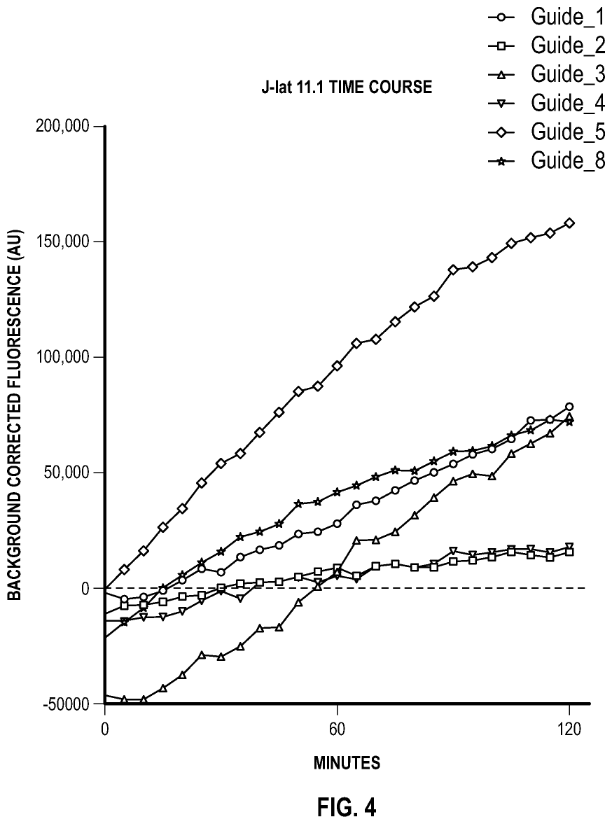

Further experimental results showed that “accessible” regions of HIV RNA are most favorable targets for crRNA: HIV RNA binding. Both cell-associated RNA (J-Lat cell lines) and supernatant RNA (ACH-2 & U-1 cell lines) were evaluated. Using RNase A, maximum signals can be detected. Importantly, not all crRNAs were equally effective as illustrated in FIGS. 3A-3D. FIG. 4 shows same data as FIG. 3B, plotted over time.

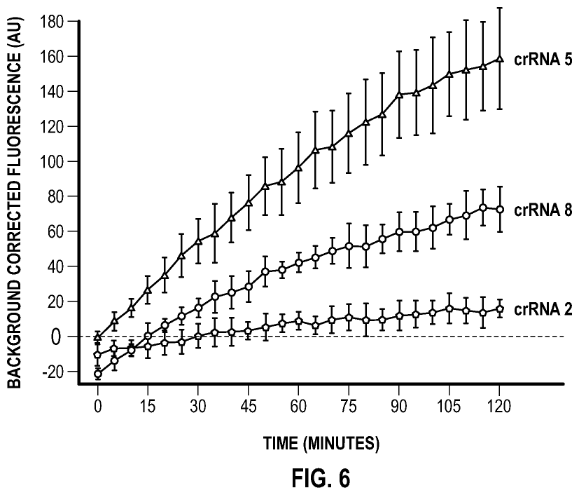

FIG. 6 shows specific detection of HIV-1 RNA with Cas13a:crRNA complexes and RNaseAlert detection reagent using three different crRNAs. The crRNAs were tested with total RNAs isolated from J-Lat 11.1 cells reactivated with TNFα (10 ng/ml). As illustrated, different crRNAs provide somewhat different results, indicating that different crRNA may have more or less access to the HIV RNA.

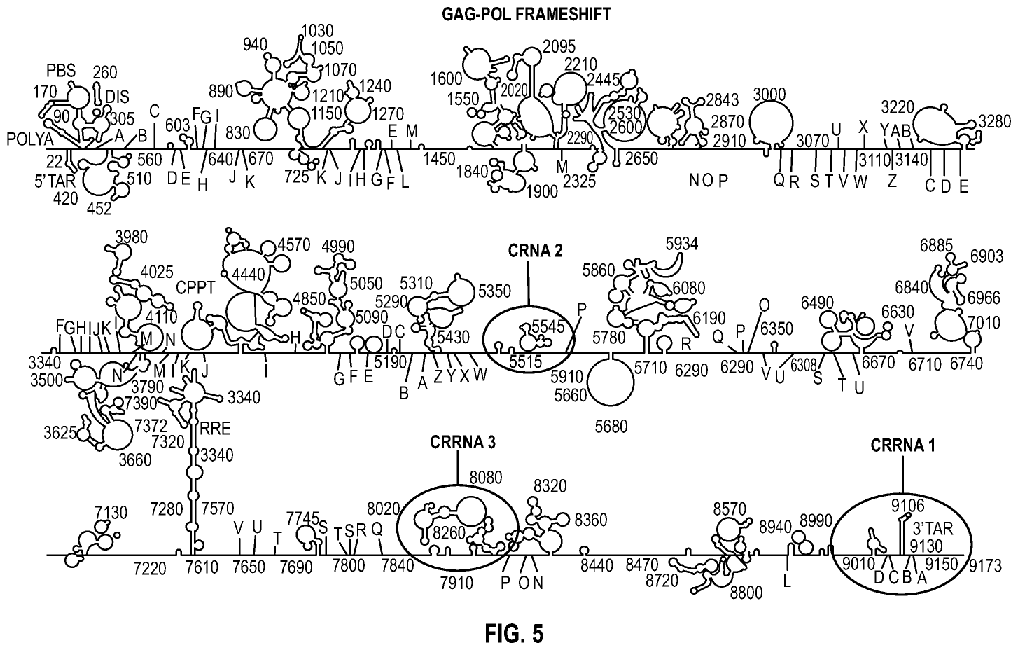

FIG. 5 shows a schematic of the HIV-1 RNA genome and the extensive secondary structure composed of many local and long-distance interactions. Also shown are different crRNA recognition sites within the HIV-1 RNA genome for the crRNAs used for the detection of HIV shown in FIG. 6.

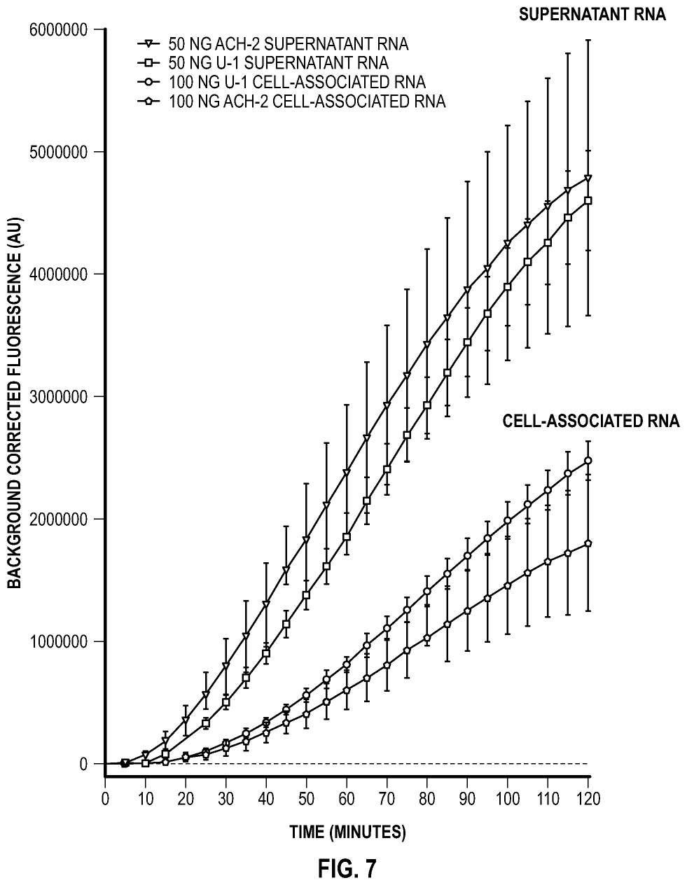

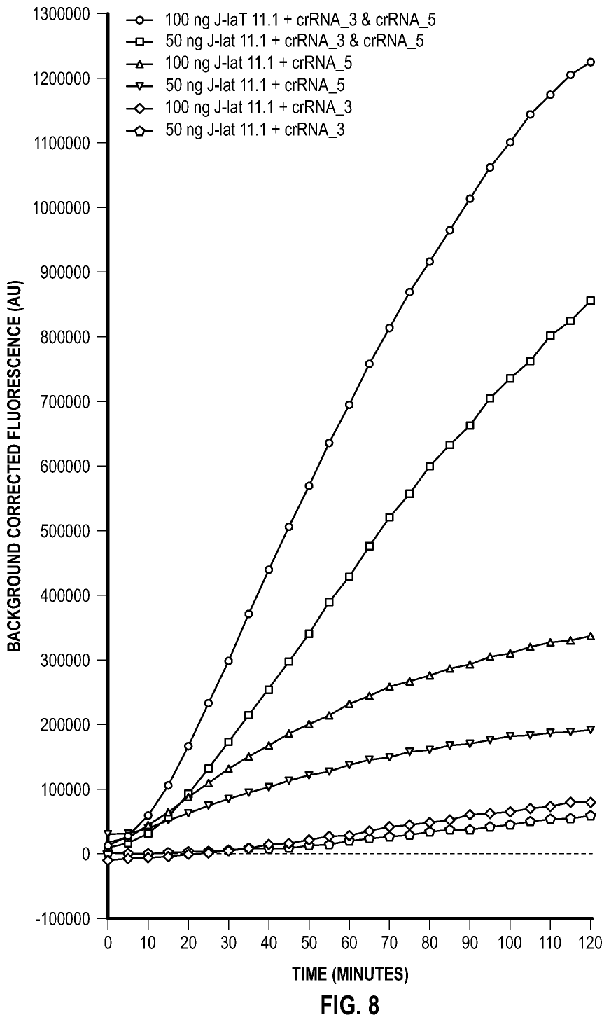

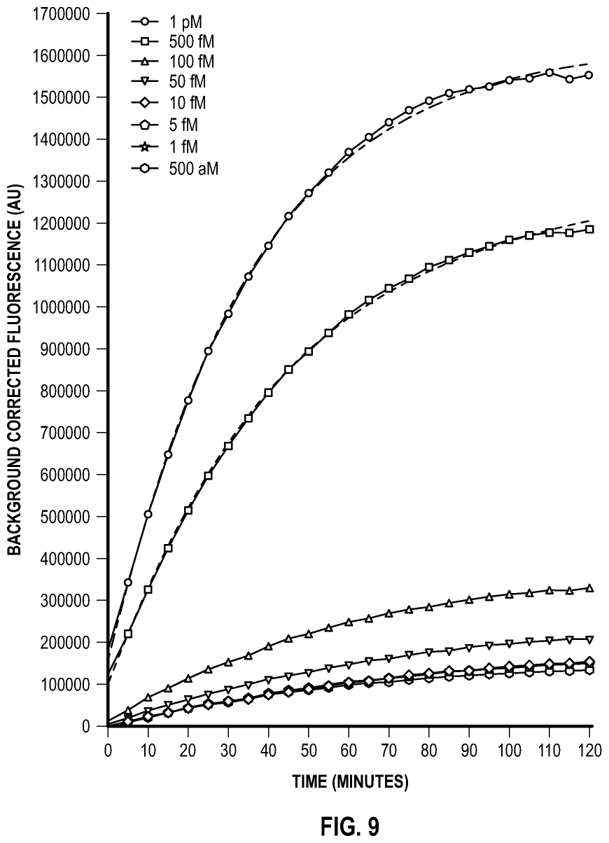

FIG. 7 shows that supernatant (or virion) RNA is more easily detected with lower RNA input compared to cell-associated RNA from two replication competent cell lines, ACH-2 and U-1 cell lines. FIG. 8 shows that the use of multiple crRNAs (multiplexing) can improve detection of HIV-1 RNA. FIG. 9 shows that Cas13a:crRNA

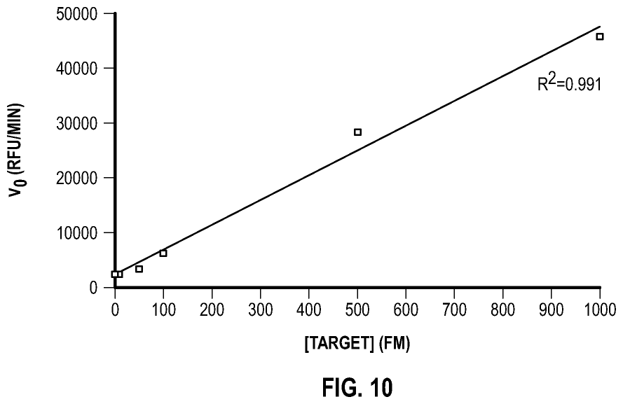

RNP complexes can detect target RNA (in this instance, a 50-nucleotide synthetic ssRNA containing a complimentary sequence to the crRNA) down to 500 aM, or 3.01×10⁵ copies/mL. The initial rates at the x-intercept of FIG. 9 were calculated for each concentration of “target” RNA and plotted (FIG. 10). Linear regression allows for a best fit line to be calculated, which can be used as a standard curve to interpolate concentrations of unknown samples.

### Example 2: Optimized CRISPR RNA (crRNA) Selection and Cas13a Protein

Cas13a activity is governed by target accessibility as the secondary structure of the target RNA influences the ability of the Cas13a:crRNA complex to bind the target sites. The HIV-1 RNA transcript contains several well-characterized RNA elements, and more recent studies have mapped the secondary structure of the entire HIV-1 genome at single nucleotide resolution (see, e.g. FIG. 5). These studies have shown extensive RNA secondary structure, in addition to long-range inter-genomic interactions, which will inform design of HIV-1 specific crRNAs.

To optimize accessibility and minimize hindrance by structure, crRNA spacer sequences will be synthesized across regions of the HIV-1 RNA genome identified as highly conserved among isolates. Comprehensive sequence information for many subtypes is available from the Los Alamos National Laboratory (LANL) HIV Sequence Database. This data will be analyzed to identify regions of conservation and low secondary structure for crRNA design and synthesis (Synthego). Conservation among the 3352 HIV isolates currently listed in the LANL HIV database will be calculated using the “Consensus Maker” tool, and further verified via multiple sequence alignment program ClustalX. Tiling of spacers will include a 5-nt overlap and will be designed in 20-nt intervals. Of special interest are regions used for primers previously used in RT-PCR including, if available, in FDA-approved detection kits.

The crRNAs will be pre-incubated with LbuCas13a protein and then with either the synthetic target HIV RNAs or isolated RNAs from infected and uninfected cells. Other Cas13 homologs will also be tested. RNA isolated from cell supernatant from ACH-2 and U1 cell lines, which contain HIV virions after activation with TNFa, to phenocopy detection of circulating viruses in the blood will be included. Samples will be analyzed on a Spectramax plate reader. Normalized reporter signal curve time courses will be fitted with single-exponential decays and the apparent rates will be compared with a standard curve of defined activator RNA concentrations. RNA from uninfected cells or mismatched synthetic target RNAs will be included as controls and the findings will be compared to parallel RT-qPCR and ddPCR results from the same samples.

LbuCas13a is a robust multiple-turnover enzyme capable of at least 10⁴ turnovers per target RNA recognized, even in the presence of low target RNA concentrations. This indicates that crRNA-directed trans cleavage is potent and detectable even at low levels of activated protein. Eleven homologs of Cas13a have been cloned, purified, and tested for the following characteristics: trans-ssRNA cleavage rate, sensitivity for HIV-1 RNA, and false-positive rate as a result of aberrant or nonspecific activation of the complex, all in the context of background RNA. These homologs will be tested with the crRNAs described above and all identified crRNAs will also be tested, alone or in combination. The Cas13a proteins to be tested are from the following bacteria: Leptotrichia wadei, Rhodobacter capsulatus, Herbinix hemicellulosilytica, Leptotrichia buccalis, Listeria seeligeri, Paludibacter propionicigenes, Lachnospiraceae bacterium, [Eubacterium] rectale, Listeria newyorkensis, Clostridium aminophilum, Leptotrichia shahii. Open reading frames of Cas13a proteins are cloned and available for bacterial expression and purification as described in East-Seletsky Nature (2016) and East-Seletsky Molecular Cell (2017).

For example, a Leptotrichia wadei Cas13a endonuclease can have the following sequence (SEQ ID NO:10; NCBI accession no. WP_036059678.1).

| | |
|---|---|
| 1 | MKITKIDGVS HYKKQDKGIL KKKWKDLDER KQREKIEARY |
| 41 | NKQIESKIYK EFFRLKNKKR IEKEEDQNIK SLYFFIKELY |
| 81 | LNEKNEEWEL KNINLEILDD KERVIKGYKF KEDVYFFKEG |
| 121 | YKEYYLRILF NNLIEKVQNE NREKVRKNKE FLDLKEIFKK |
| 161 | YKNRKIDLLL KSINNNKINL EYKKENVNEE IYGINPTNDR |
| 201 | EMTFYELLKE IIEKKDEQKS ILEEKLDNFD ITNFLENIEK |
| 241 | IFNEETEINI IKGKVLNELR EYIKEKEENN SDNKLKQIYN |
| 281 | LELKKYIENN FSYKKQKSKS KNGKNDYLYL NFLKKIMFIE |
| 321 | EVDEKKEINK EKFKNKINSN FKNLFVQHIL DYGKLLYYKE |
| 361 | NDEYIKNTGQ LETKDLEYIK TKETLIRKMA VLVSFAANSY |
| 401 | YNLFGRVSGD ILGTEVVKSS KTNVIKVGSH IFKEKMLNYF |
| 441 | FDFEIFDANK IVEILESISY SIYNVRNGVG HFNKLILGKY |
| 481 | KKKDINTNKR IEEDLNNNEE IKGYFIKKRG EIERKVKEKF |
| 521 | LSNNLQYYYS KEKIENYFEV YEFEILKRKI PFAPNFKRII |
| 561 | KKGEDLFNNK NNKKYEYFKN FDKNSAEEKK EFLKTRNFLL |
| 601 | KELYYNNFYK EFLSKKEEFE KIVLEVKEEK KSRGNINNKK |
| 641 | SGVSFQSIDD YDTKINISDY IASIHKKEME RVEKYNEEKQ |
| 681 | KDTAKYIRDF VEEIFLTGFI NYLEKDKRLH FLKEEFSILC |
| 721 | NNNNNVVDFN ININEEKIKE FLKENDSKTL NLYLFFNMID |
| 761 | SKRISEFRNE LVKYKQFTKK RLDEEKEFLG IKIELYETLI |
| 801 | EFVILTREKL DTKKSEEIDA WLVDKLYVKD SNEYKEYEEI |
| 841 | LKLFVDEKIL SSKEAPYYAT DNKTPILLSN FEKTRKYGTQ |
| 881 | SFLSEIQSNY KYSKVEKENI EDYNKKEEIE QKKKSNIEKL |
| 921 | QDLKVELHKK WEQNKITEKE IEKYNNTTRK INEYNYLKNK |
| 961 | EELQNVYLLH EMLSDLLARN VAFFNKWERD FKFIVIAIKQ |
| 1001 | FLRENDKEKV NEFLNPPDNS KGKKVYFSVS KYKNTVENID |
| 1041 | GIHKNFMNLI FLNNKEMNRK IDKMNCAIWV YFRNYIAHFL |
| 1081 | HLHTKNEKIS LISQMNLLIK LFSYDKKVQN HILKSTKTLL |
| 1121 | EKYNIQINFE ISNDKNEVFK YKIKNRLYSK KGKMLGKNNK |
| 1161 | LENEFLE NVKAMLEYSE |

Other sequences for Leptotrichia wadei Cas13a endonucleases are also available, such as those NCBI accession nos. BBM46759.1, BBM48616.1, BBM48974.1, BBM48975.1, and WP_021746003.1.

In another example, a Herbinix hemicellulosilytica Cas13a endonuclease can have the following sequence (SEQ ID NO:11; NCBI accession no. WP_103203632.1).

| | |
|---|---|
| 1 | MKLTRRRISG NSVDQKITAA FYRDMSQGLL YYDSEDNDCT |
| 41 | DKVIESMDFE RSWRGRILKN GEDDKNPFYM FVKGLVGSND |
| 81 | KIVCEPIDVD SDPDNLDILI NKNLTGFGRN LKAPDSNDTL |
| 121 | ENLIRKIQAG IPEEEVLPEL KKIKEMIQKD IVNRKEQLLK |
| 161 | SIKNNRIPFS LEGSKLVPST KKMKWLFKLI DVPNKTFNEK |
| 201 | MLEKYWEIYD YDKLKANITN RLDKTDKKAR SISRAVSEEL |
| 241 | REYHKNLRTN YNRFVSGDRP AAGLDNGGSA KYNPDKEEFL |
| 281 | LFLKEVEQYF KKYFPVKSKH SNKSKDKSLV DKYKNYCSYK |
| 321 | VVKKEVNRSI INQLVAGLIQ QGKLLYYFYY NDTWQEDFLN |
| 361 | SYGLSYIQVE EAFKKSVMTS LSWGINRLTS FFIDDSNTVK |
| 401 | FDDITTKKAK EAIESNYFNK LRTCSRMQDH FKEKLAFFYP |
| 441 | VYVKDKXDRP DDDIENLIVL VKNAIESVSY LRNRTFHFKE |
| 481 | SSLLELLKEL DDKNSGQNKI DYSVAAEFIK RDIENLYDVF |
| 521 | REQIRSLGIA EYYKADMISD CFKTCGLEFA LYSPKNSLMP |
| 561 | AFKNVYKRGA NLNKAYIRDK GPKETGDQGQ NSYKALEEYR |
| 601 | ELTWYIEVKN NDQSYNAYKN LLQLIYYHAF LPEVRENEAL |
| 641 | ITDFINRTKE WNRKETEERL NTKNNKKHKN FDENDDITVN |
| 681 | TYRYESIPDY QGESLDDYLK VLQRKQMARA KEVNEKEEGN |
| 721 | NNYIQFIRDV VVWAFGAYLE NKLKNYKNEL QPPLSKENIG |
| 761 | LNDTLKELFP EEKVKSPFNI KCRFSISTFI DNKGKSTDNT |
| 801 | SAEAVKTDGK EDEKDKKNIK RKDLLCFYLF LRLLDENEIC |
| 841 | KLQHQFIKYR CSLKERRFPG NRTKLEKETE LLAELEELME |
| 881 | LVRFTMPSIP EISAKAESGY DTMIKKYFKD FIEKKVFKNP |
| 921 | KTSNLYYHSD SKTPVTRKYM ALLMRSAPLH LYKDIFKGYY |
| 961 | LITKKECLEY IKLSNIIKDY QNSLNELHEQ LERIKLKSEK |
| 1001 | QNGKDSLYLD KKDFYKVKEY VENLEQVARY KHLQHKINFE |
| 1041 | SLYRIFRIHV DIAARMVGYT QDWERDMHFL FKALVYNGVL |
| 1081 | EERRFEAIFN NNDDNNDGRI VKKIQNNLNN KNRELVSMLC |
| 1121 | WNKKLNKNEF GAIIWKRNPI AHLNHFTQTE QNSKSSLESL |
| 1161 | INSLRILLAY DRKRQNAVTK TINDLLLNDY HIRIKWEGRV |
| 1201 | DEGQIYFNIK EKEDIENEPI IHLKHLHKKD CYIYKNSYMF |
| 1241 | DKQKEWICNG IKEEVYDKSI LKCIGNLFKF DYEDKNKSSA |
| 1281 | NPKHT |

For example, a Leptotrichia buccalis Cas13a endonuclease can have the following sequence (SEQ ID NO:12; NCBI accession no. WP_015770004.1).

| | |
|---|---|
| 1 | MKVTKVGGIS HKKYTSEGRL VKSESEENRT DERLSALLNM |
| 41 | RLDMYIKNPS STETKENQKR IGKLKKFFSN KMVYLKDNTL |
| 81 | SLKNGKKENI DREYSETDIL ESDVRDKKNF AVLKKIYLNE |
| 121 | NVNSEELEVF RNDIKKKLNK INSLKYSFEK NKANYQKINE |
| 161 | NNIEKVEGKS KRNIIYDYYR ESAKRDAYVS NVKEAFDKLY |
| 201 | KEEDIAKLVL EIENLTKLEK YKIREFYHEI IGRKNDKENF |
| 241 | AKIIYEEIQN VNNMKELIEK VPDMSELKKS QVFYKYYLDK |
| 281 | EELNDKNIKY AFCHFVEIEM SQLLKNYVYK RLSNISNDKI |
| 321 | KRIFEYQNLK KLIENKLLNK LDTYVRNCGK YNYYLQDGEI |
| 361 | ATSDFIARNR QNEAFLRNII GVSSVAYFSL RNILETENEN |
| 401 | DITGRMRGKT VKNNKGEEKY VSGEVDKIYN ENKKNEVKEN |
| 441 | LKMFYSYDFN MDNKNEIEDF FANIDEAISS IRHGIVHFNL |
| 481 | ELEGKDIFAF KNIAPSEISK KMFQNEINEK KLKLKIFRQL |
| 521 | NSANVFRYLE KYKILNYLKR TRFEFVNKNI PFVPSFTKLY |
| 561 | SRIDDLKNSL GIYWKTPKTN DDNKTKEIID AQIYLLKNIY |
| 601 | YGEFLNYFMS NNGNFFEISK EIIELNKNDK RNLKTGFYKL |
| 641 | QKFEDIQEKI PKEYLANIQS LYMINAGNQD EEEKDTYIDF |
| 681 | IQKIFLKGFM TYLANNGRLS LIYIGSDEET NTSLAEKKQE |
| 721 | FDKFLKKYEQ NNNIKIPYEI NEFLREIKLG NILKYTERLN |
| 761 | MFYLILKLLN HKELTNLKGS LEKYQSANKE EAFEDQLELI |
| 801 | NLLNLDNNRV TEDFELEADE IGKFLDFNGN KVKDNKELKK |
| 841 | FDTNKIYFDG ENIIKHRAFY NIKKYGMLNL LEKIADKAGY |
| 881 | KISIEELKKY SNKKNEIEKN HKMQENLHRK YARPRKDEKF |
| 921 | TDEDYESYKQ AIENIEEYTH LKNKVEFNEL NLLQGLLLRI |
| 961 | LHRLVGYTSI WERDLRFRLK GEFPENQYIE EIFNFENKKN |
| 1001 | VKYKGGQIVE KYIKFYKELH QNDEVKINKY SSANIKVLKQ |
| 1041 | EKKDLYIRNY IAHFNYIPHA EISLLEVLEN LRKLLSYDRK |
| 1081 | LKNAVMKSVV DILKEYGFVA TFKIGADKKI GIQTLESEKI |
| 1121 | VHLKNLKKKK LMTDRNSEEL CKLVKIMFEY KMEEKKSEN |

For example, a Leptotrichia seeligeri Cas13a endonuclease can have the following sequence (SEQ ID NO:13; NCBI accession no. WP_012985477.1).

| | |
|---|---|
| 1 | MWISIKTLIH HLGVLFFCDY MYNRREKKII EVKTMRITKV |
| 41 | EVDRKKVLIS RDKNGGKLVY ENEMQDNTEQ IMHHKKSSFY |
| 81 | KSVVNKTICR PEQKQMKKLV HGLLQENSQE KIKVSDVTKL |
| 121 | NISNFLNHRF KKSLYYFPEN SPDKSEEYRI EINLSQLLED |
| 161 | SLKKQQGTFI CWESFSKDME LYINWAENYI SSKTKLIKKS |
| 201 | IRNNRIQSTE SRSGQLMDRY MKDILNKNKP FDIQSVSEKY |
| 241 | QLEKLTSALK ATFKEAKKND KEINYKLKST LQNHERQIIE |
| 281 | ELKENSELNQ FNIEIRKHLE TYFPIKKTNR KVGDIRNLEI |
| 321 | GEIQKIVNHR LKNKIVQRIL QEGKLASYEI ESTVNSNSLQ |
| 361 | KIKIEEAFAL KFINACLFAS NNLRNMVYPV CKKDILMIGE |
| 401 | FKNSFKEIKH KKFIRQWSQF FSQEITVDDI ELASWGLRGA |
| 441 | IAPIRNEIIH LKKHSWKKFF NNPTFKVKKS KIINGKTKDV |
| 481 | TSEFLYKETL FKDYFYSELD SVPELIINKM ESSKILDYYS |
| 521 | SDQLNQVFTI PNFELSLLTS AVPFAPSFKR VYLKGFDYQN |
| 561 | QDEAQPDYNL KLNIYNEKAF NSEAFQAQYS LFKMVYYQVF |
| 601 | LPQFTTNNDL FKSSVDFILT LNKERKGYAK AFQDIRKMNK |
| 641 | DEKPSEYMSY IQSQLMLYQK KQEEKEKINH FEKFINQVFI |
| 681 | KGFNSFIEKN RLTYICHPTK NTVPENDNIE IPFHTDMDDS |
| 721 | NIAFWLMCKL LDAKQLSELR NEMIKFSCSL QSTEEISTFT |
| 761 | KAREVIGLAL LNGEKGCNDW KELFDDKEAW KKNMSLYVSE |
| 801 | ELLQSLPYTQ EDGQTPVINR SIDLVKKYGT ETILEKLFSS |
| 841 | SDDYKVSAKD IAKLHEYDVT EKIAQQESLH KQWIEKPGLA |
| 881 | RDSAWTKKYQ NVINDISNYQ WAKTKVELTQ VRHLHQLTID |
| 921 | LLSRLAGYMS IADRDFQFSS NYILERENSE YRVTSWILLS |
| 961 | ENKNKNKYND YELYNLKNAS IKVSSKNDPQ LKVDLKQLRL |
| 1001 | TLEYLELFDN RLKEKRNNIS HFNYLNGQLG NSILELFDDA |
| 1041 | RDVLSYDRKL KNAVSKSLKE ILSSHGMEVT FKPLYQTNHH |
| 1081 | LKIDKLQPKK IHHLGEKSTV SSNQVSNEYC QLVRTLLTMK |

For example, a Paludibacter propionicigenes Cas13a endonuclease can have the following sequence (SEQ ID NO:14, NCBI accession no. WP_013443710.1).

| | |
|---|---|
| 1 | MRVSKVKVKD GGKDKMVLVH RKTTGAQLVY SGQPVSNETS |
| 41 | NILPEKKRQS FDLSTLNKTI IKFDTAKKQK LNVDQYKIVE |
| 81 | KIFKYPKQEL PKQIKAEEIL PFLNHKFQEP VKYWKNGKEE |
| 121 | SFNLTLLIVE AVQAQDKRKL QPYYDWKTWY IQTKSDLLKK |
| 161 | SIENNRIDLT ENLSKRKKAL LAWETEFTAS GSIDLTHYHK |
| 201 | VYMTDVLCKM LQDVKPLTDD KGKINTNAYH RGLKKALQNH |
| 241 | QRAIFGTREV PNEANRADNQ LSIYHLEVVK YLEHYFPIKT |
| 281 | SKRRNTADDI AHYLKAQTLK TTIEKQLVNA IRANIIQQGK |
| 321 | TNHHELKADT TSNDLIRIKT NEAFVLNLTG TCAFAANNIR |
| 361 | NMVDNEQTND ILGKGDFIKS LLKDNINSQL YSFFFGEGLS |
| 401 | TNKAEKETQL WGIRGAVQQI RNNVNHYKKD ALKTVFNISN |
| 441 | FENPTITDPK QQTNYADTIY KARFINELEK IPEAFAQQLK |
| 481 | TGGAVSYYTI ENLKSLLTTF QFSLCRSTIP FAPGFKKVFN |
| 521 | GGINYQNAKQ DESFYELMLE QYLRKENFAE ESYNARYFML |
| 561 | KLIYNNLFLP GFTTDRKAFA DSVGFVQMQN KKQAEKVNPR |
| 601 | KKEAYAFEAV RPMTAADSIA DYMAYVQSEL MQEQNKKEEK |
| 641 | VAEETRINFE KFVLQVFIKG FDSFLRAKEF DFVQMPQPQL |
| 681 | TATASNQQKA DKLNQLEASI TADCKLTPQY AKADDATHIA |
| 721 | FYVFCKLLDA AHLSNLRNEL IKFRESVNEF KFHHLLEIIE |
| 761 | ICLLSADVVP TDYRDLYSSE ADCLARLRPF IEQGADITNW |
| 801 | SDLFVQSDKH SPVIHANIEL SVKYGTTKLL EQIINKDTQF |
| 841 | KTTEANFTAW NTAQKSIEQL IKQREDHHEQ WVKAKNADDK |
| 881 | EKQERKREKS NFAQKFIEKH GDDYLDICDY INTYNWLDNK |
| 921 | MHFVHLNRLH GLTIELLGRM AGFVALFDRD FQFFDEQQIA |
| 961 | DEFKLHGFVN LHSIDKKLNE VPTKKIKEIY DIRNKIIQIN |
| 1001 | GNKINESVRA NLIQFISSKR NYYNNAFLHV SNDEIKEKQM |
| 1041 | YDIRNHIAHF NYLTKDAADF SLIDLINELR ELLHYDRKLK |
| 1081 | NAVSKAFIDL FDKHGMILKL KLNADHKLKV ESLEPKKIYH |
| 1121 | LGSSAKDKPE YQYCTNQVMM AYCNMCRSLL EMKK |

For example, a Lachnospiraceae bacterium Cas 13a endonuclease can have the following sequence (SEQ ID NO: IS; NCBI accession no. WP_022785443. 1).

| | |
|---|---|
| 1 | MKISKVREEN RGAKLTVNAK TAVVSENRSQ EGILYNDPSR |
| 41 | YGKSRKNDED RDRYIESRLK SSGKLYRIFN EDKNKRETDE |
| 81 | LQWFLSEIVK KINRRNGLVL SDMLSVDDRA FEKAFEKYAE |
| 121 | LSYTNRRNKV SGSPAFETCG VDAATAERLK GIISETNFIN |
| 161 | RIKNNIDNKV SEDIIDRIIA KYLKKSLCRE RVKRGLKKLL |
| 201 | MNAFDLPYSD PDIDVQRDFI DYVLEDFYHV RAKSQVSRSI |
| 241 | KNMNMPVQPE GDGKFAITVS KGGTESGNKR SAEKEAFKKF |
| 281 | LSDYASLDER VRDDMLRRMR RLVVLYFYGS DDSKLSDVNE |
| 321 | KFDVWEDHAA RRVDNREFIK LPLENKLANG KTDKDAERIR |
| 361 | KNTVKELYRN QNIGCYRQAV KAVEEDNNGR YFDDKMLNMF |
| 401 | FIHRIEYGVE KIYANLKQVT EFKARTGYLS EKIWKDLINY |
| 441 | ISIKYIAMGK AVYNYAMDEL NASDKKEIEL GKISEEYLSG |
| 481 | ISSFDYELIK AEEMLQRETA VYVAFAARHL SSQTVELDSE |
| 521 | NSDFLLLKPK GTMDKNDKNK LASNNILNFL KDKETLRDTI |
| 561 | LQYFGGHSLW TDFPFDKYLA GGKDDVDFLT DLKDVIYSMR |
| 601 | NDSFHYATEN HNNGKWNKEL ISAMFEHETE RMTVVMKDKF |
| 641 | YSNNLPMFYK NDDLKKLLID LYKDNVERAS QVPSFNKVFV |
| 681 | RKNFPALVRD KDNLGIELDL KADADKGENE LKFYNALYYM |
| 721 | FKEIYYNAFL NDKNVRERFI TKATKVADNY DRNKERNLKD |
| 761 | RIKSAGSDEK KKLREQLQNY IAENDFGQRI KNIVQVNPDY |
| 801 | TLAQICQLIM TEYNQQNNGC MQKKSAARKD INKDSYQHYK |
| 841 | MLLLVNLRKA FLEFIKENYA FVLKPYKHDL CDKADFVPDF |
| 881 | AKYVKPYAGL ISRVAGSSEL QKWYIVSRFL SPAQANHMLG |
| 921 | FLHSYKQYVW DIYRRASETG TEINHSIAED KIAGVDITDV |
| 961 | DAVIDLSVKL CGTISSEISD YFKDDEVYAE YISSYLDFEY |
| 1001 | DGGNYKDSLN RFCNSDAVND QKVALYYDGE HPKLNRNIIL |
| 1041 | SKLYGERRFL EKITDRVSRS DIVEYYKLKK ETSQYQTKGI |
| 1081 | FDSEDEQKNI KKFQEMKNIV EFRDLMDYSE IADELQGQLI |
| 1121 | NWIYLRERDL MNFQLGYHYA CLNNDSNKQA TYVTLDYQGK |
| 1161 | KNRKINGAIL YQICAMYING LPLYYVDKDS SEWTVSDGKE |
| 1201 | STGAKIGEFY RYAKSFENTS DCYASGLEIF ENISEHDNIT |
| 1241 | ELRNYIEHFR YYSSFDRSFL GIYSEVFDRF FTYDLKYRKN |
| 1281 | VPTILYNILL QHFVNVRFEF VSGKKMIGID KKDRKIAKEK |
| 1321 | ECARITIREK NGVYSEQFTY KLKNGTVYVD ARDKRYLQSI |
| 1361 | IRLLFYPEKV NMDEMIEVKE KKKPSDNNTG KGYSKRDRQQ |
| 1401 | DRKEYDKYKE KKKKEGNFLS GMGGNINWDE INAQLKN |

For example, a Leptotrichia shahii Cas13a endonuclease can have the following sequence (SEQ ID NO:16; NCBI accession no. BBM39911.1).

| | |
|---|---|
| 1 | MGNLFGHKRW YEVRDKKDFK IKRKVKVKRN YDGNKYILNI |
| 41 | NENNNKEKID NNKFIRKYIN YKKNDNILKE FTRKFHAGNI |
| 81 | LFKLKGKEGI IRIENNDDFL ETEEVVLYIE AYGKSEKLKA |
| 121 | LGITKKKIID EAIRQGITKD DKKIEIKRQE NEEEIEIDIR |
| 161 | DEYTNKTLND CSIILRIIEN DELETKKSIY EIFKNINMSL |
| 201 | YKIIEKIIEN ETEKVFENRY YEEHLREKLL KDDKIDVILT |
| 241 | NFMEIREKIK SNLEILGFVK FYLNVGGDKK KSKNKKMLVE |
| 281 | KILNINVDLT VEDIADFVIK ELEFWNITKR IEKVKKVNNE |
| 321 | FLEKRRNRTY IKSYVLLDKH EKFKIERENK KDKIVKFFVE |
| 361 | NIKNNSIKEK IEKILAEFKI DELIKKLEKE LKKGNCDTEI |
| 401 | FGIFKKHYKV NFDSKKFSKK SDEEKELYKI IYRYLKGRIE |
| 441 | KILVNEQKVR LKKMEKIEIE KILNESILSE KILKRVKQYT |
| 481 | LEHIMYLGKL RHNDIDMTTV NTDDFSRLHA KEELDLELIT |
| 521 | FFASTNMELN KIFSRFNINN DENIDFFGGD REKNYVLDKK |
| 561 | ILNSKIKIIR DLDFIDNKNN ITNNFIRKFT KIGTNERNRI |
| 601 | LHAISKERDL QGTQDDYNKV INIIQNLKIS DEEVSKALNL |
| 641 | DVVFKDKKNI ITKINDIKIS EENNNDIKYL PSFSKVLPEI |
| 681 | LNLYRNNPKN EPFDTIETEK IVLNALIYVN KELYKKLILE |
| 721 | DDLEENESKN IFLQELKKTL GNIDEIDENI IENYYKNAQI |
| 761 | SASKGNNKAI KKYQKKVIEC YIGYLRKNYE ELFDFSDFKM |
| 801 | NIQEIKKQIK DINDNKTYER ITVKTSDKTI VINDDFEYII |
| 841 | SIFALLNSNA VINKIRNRFF ATSVWLNTSE YQNIIDILDE |
| 881 | IMQLNTLRNE CITENWNLNL EEFIQKMKEI EKDFDDFKIQ |
| 921 | TKKEIFNNYY EDIKNNILTE FKDDINGCDV LEKKLEKIVI |
| 961 | FDDETKFEID KKSNILQDEQ RKLSNINKKD LKKKVDQYIK |
| 1001 | DKDQEIKSKI LCRIIFNSDF LKKYKKEIDN LIEDMESENE |
| 1041 | NKFQEIYYPK ERKNELYIYK KNLFLNIGNP NEDKIYGLIS |
| 1081 | NDIKMADAKF LFNIDGKNIR KNKISEIDAI LKNINDKLNG |
| 1121 | YSKEYKEKYI KKLKENDDFF AKNIQNKNYK SFEKDYNRVS |
| 1161 | EYKKIRDLVE FNYLNKIESY LIDINWKLAI QMARFERDMH |
| 1201 | YIVNGLRELG IIKLSGYNTG ISRAYPKRNG SDGFYTTTAY |
| 1241 | YKFFDEESYK KFEKICYGFG IDLSENSEIN KPENESIRNY |
| 1281 | ISHFYIVRNP FADYSIAEQI DRVSNLLSYS TRYNNSTYAS |
| 1321 | VFEVFKKDVN LDYDELKKKF KLIGNNDILE RLMKPKKVSV |
| 1361 | LELESYNSDY IKNLIIELLT KIENTNDTL |

40)

In another example, a Leptotrichia buccalis C-1013-b Cas13a endonuclease can have the following sequence (SEQ ID NO: 17. NCBI accession no. C7NBY4; AltName

LbuC2c2).

| | |
|---|---|
| 1 | MKVTKVGGIS HKKYTSEGRL VKSESEENRT DERLSALLNM |
| 41 | RLDMYIKNPS STETKENQKR IGKLKKFFSN KMVYLKDNTL |
| 81 | SLKNGKKENI DREYSETDIL ESDVRDKKNF AVLKKIYLNE |
| 121 | NVNSEELEVF RNDIKKKLNK INSLKYSFEK NKANYQKINE |
| 161 | NNIEKVEGKS KRNIIYDYYR ESAKRDAYVS NVKEAFDKLY |
| 201 | KEEDIAKLVL EIENLTKLEK YKIREFYHEI IGRKNDKENF |
| 241 | AKIIYEEIQN VNNMKELIEK VPDMSELKKS QVFYKYYLDK |
| 281 | EELNDKNIKY AFCHFVEIEM SQLLKNYVYK RLSNISNDKI |
| 321 | KRIFEYQNLK KLIENKLLNK LDTYVRNCGK YNYYLQDGEI |
| 361 | ATSDFIARNR QNEAFLRNII GVSSVAYFSL RNILETENEN |
| 401 | DITGRMRGKT VKNNKGEEKY VSGEVDKIYN ENKKNEVKEN |
| 441 | LKMFYSYDFN MDNKNEIEDF FANIDEAISS IRHGIVHFNL |
| 481 | ELEGKDIFAF KNIAPSEISK KMFQNEINEK KLKLKIFRQL |
| 521 | NSANVFRYLE KYKILNYLKR TRFEFVNKNI PFVPSFTKLY |
| 561 | SRIDDLKNSL GIYWKTPKTN DDNKTKEIID AQIYLLKNIY |
| 601 | YGEFLNYFMS NNGNFFEISK EIIELNKNDK RNLKTGFYKL |
| 641 | QKFEDIQEKI PKEYLANIQS LYMINAGNQD EEEKDTYIDF |
| 681 | IQKIFLKGFM TYLANNGRLS LIYIGSDEET NTSLAEKKQE |
| 721 | FDKFLKKYEQ NNNIKIPYEI NEFLREIKLG NILKYTERLN |
| 761 | MFYLILKLLN HKELTNLKGS LEKYQSANKE EAFSDQLELI |
| 801 | NLLNLDNNRV TEDFELEADE IGKFLDFNGN KVKDNKELKK |
| 841 | FDTNKIYFDG ENIIKHRAFY NIKKYGMLNL LEKIADKAGY |
| 881 | KISIEELKKY SNKKNEIEKN HKMQENLHRK YARPRKDEKF |
| 921 | TDEDYESYKQ AIENIEEYTH LKNKVEFNEL NLLQGLLLRI |
| 961 | LHRLVGYTSI WERDLRFRLK GEFPENQYIE EIFNFENKKN |
| 1001 | VKYKGGQIVE KYIKFYKELH QNDEVKINKY SSANIKVLKQ |
| 1041 | EKKDLYIRNY IAHFNYIPHA EISLLEVLEN LRKLLSYDRK |
| 1081 | LKNAVMKSVV DILKEYGFVA TFKIGADKKI GIQTLESEKI |
| 1121 | VHLKNLKKKK LMTDRNSEEL CKLVKIMFEY KMEEKKSEN |

Purified proteins will first be assayed for trans-ssRNA cleavage rates with HIV-specific crRNAs. This rate measures the trans cleavage of the fluorophore quencher-labeled ssRNA, which serves as an outread for complex activation and as a surrogate for the presence of HIV RNA. Notably, the rate at which trans cleavage reaches saturation varies greatly among Cas13a homologs. If the trans rate is too low, fluorescence outread will be undetectable, especially in the context of an excess of unlabeled human RNA. To systematically study the rate of trans cleavage in this context, we will test the ability of a preassembled ternary complex comprising the Cas13a:crRNA ribonucleoprotein (RNP) complex plus a bound synthetic ssRNA activator to degrade in trans the fluorophore quencher-labeled RNaseAlert substrate in the context of increasing amounts of tRNAs or purified human non-targeting RNAs. How the rate of trans cleavage reaches saturation over time will be monitored to identify ideal homologs with the fastest rate. Variables tested in this assay include concentrations of the Cas13a:crRNA RNP and concentrations of the reporter RNA to achieve optimized rates.

Next, the sensitivity of the homologs for cis cleavage of activating HIV ssRNA in the context of competitor RNA will be analyzed. A broad range of sensitivities (˜10⁷ fold) exist for these homologs in the context of just isolated activator RNA, but the influence of additional non-targeting RNAs on the cis cleavage rate is unknown. Background RNA, especially at high concentrations, can inhibit access to HIV RNA, precluding activation of the Cas13a:crRNA complex and downstream trans-cleavage. To test the influence of background RNA on cis-cleavage, a high-throughput screen will be used. For each Cas13a homolog, dilutions of the complementary fluorescent ssRNA activator will be systematically added with and without increasing amounts of tRNAs or purified human mRNAs and analyze cis cleavage rates of the reporter over time. Each resulting time course will allow the apparent rate of complementary target sensitivity to be calculated in the context of the defined competitor RNA background.

The specificity of the homologs will also be tested in the context of background competitor RNA to ensure that related RNA sequences cannot aberrantly or non-specifically activate the Cas13a:crRNA complex. Different Cas13a homologs tolerate different numbers of mismatches in the crRNA-target duplex. For example, Cas13a from Leptotrichia shahii (LshCas13a) is sensitive to double, but not single, mismatches in the crRNA-target duplex. Moreover, the location of these mismatches within the spacer sequence is important. For example, LshCas13a is sensitive to double mismatches in the center, or in the “seed region,” of the crRNA-target duplex, but not at the 5′ or 3′ends. It was recently discovered that LbuCas 13a has a mismatch sensitive seed region that correlates well with observations for LshCas13a and the structure of LbuCas13a and that LbuCas13a has a mismatch sensitive switch region that effectively communicates activator RNA binding to the Higher Eukaryotes and Prokaryotes Nucleotide-binding (HEPN) nuclease for activation. We have generated a comprehensive mismatch sensitivity profile for LbuCas13a.

Data suggests that the mismatch sensitivity profile of homologs is quite variable. To test this comprehensively across all homologs, each homolog will be tested with crRNAs carrying systematic variations of mismatches against a known complimentary

HIV-derived target sequence. Here, positions in the center of the spacer (positions 6 to 16) will be focused on. Double, triple, and quadruple consecutive and non-consecutive mismatches in this region of the crRNA will be generated by mutating the bases to the respective complementary base (e.g. A to U). A 50-nucleotide complimentary target RNAs will also be synthesized based on the no-mismatch crRNA sequence. A high-throughput screen will be used, and the screen mixtures will be fluorescence monitored to determine permissiveness to mismatches. Once the levels of permissiveness for each homolog with complimentary target RNA alone have been determined, the assay will be repeated in the presence of dilutions of tRNAs or human cellular RNAs to test for nonspecific activation of the complex by other RNA sequences. Homologs with some flexibility in low-number base-pair mismatches towards the target RNA will be accepted to allow for sequence variation in the HIV RNA sequence, but we aim to identify crRNA sequences and Cas13a homologs that together show the lowest aberrant activation by competitor RNAs.

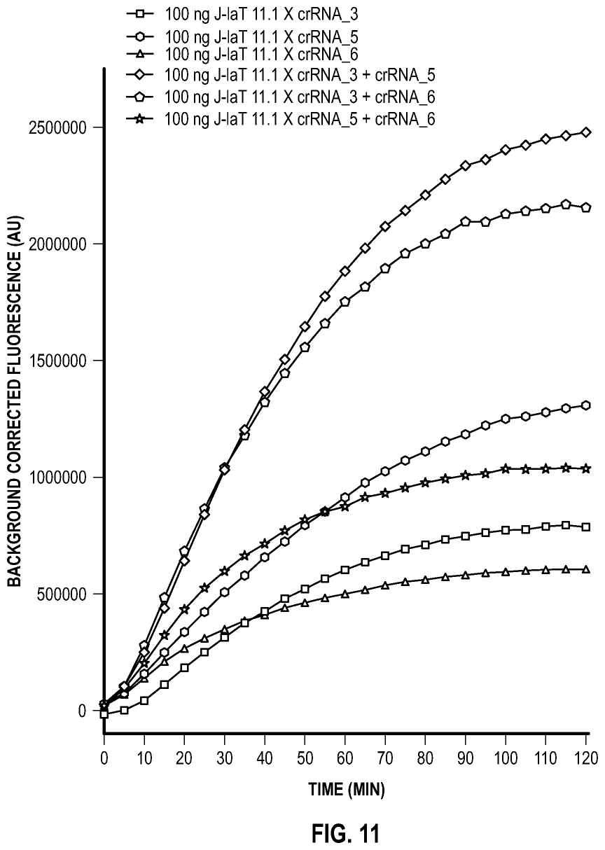

FIG. 11 demonstrates that specific combinations of crRNAs matter maximally improved the sensitivity of the assay. As shown in FIG. 11, combining RNPs using crRNA #3 and crRNA #5 or crRNA #3 and crRNA #6 give improved results relative to single crRNAs. However, combining crRNA #5 and crRNA #6 RNPs did not lead to improved sensitivity compared to using RNPs containing crRNA #5 alone. Hence, the detection limits of the assays can be optimized by selection of particular crRNAs for use in reaction mixtures with different samples.

### Example 3: Optimized Cas13a Assay for Self-Testing

Current home testing for HIV-1 has several limitations: 1) antibody-based testing utilizes saliva, where levels are generally lower than in blood; 2) this approach cannot be used for assessment of viral rebound after treatment interruptions as chronically infected individuals remain antibody-positive; and 3) advanced testing includes measurements of HIV p24 antigen but is hampered by circulating antibodies in chronically infected individuals. The gold standard for early and sensitive HIV detection is HIV RNA detection circulating in the blood, but this currently requires laboratory testing.

Here, a sensitive and single-step test for HIV RNA detection method is described that can be adapted to home testing methods that are being developed. A remaining concern is that RNases present in bodily fluids will non-specifically activate the read-out technology. As such, background fluorescence in blood, serum, and plasma samples due to circulating cellular RNases will be reduced. Cas13a and crRNA samples will be lyophilized to work towards electricity independence of the assay before rigorous analysis of cryopreserved or fresh clinical samples in comparison with FDA-approved HIV RNA testing.

Briefly, to optimize the assay for future finger-prick applications, comprehensive testing in whole blood, plasma and serum samples (without RNA extractions) obtained from uninfected blood donors will be obtained to assess and minimize background activation of the reporter readout by cellular RNases. Briefly, an RNase reporter oligonucleotide will be added directly to samples with and without dilutions of HIV-specific Cas13a:crRNA RNPs. Dilutions of purified NL4.3 HIV virions (NL4.3 generated in 293T cells after transfection) of known concentrations will then be added into each blood component and detection of HIV RNA will be measured by Cas13a:crRNA RNPs against the uninfected background. Positive detection will be confirmed and validated using RT-qPCR. If background fluorescence is found to be high in the various blood components and is not easily distinguishable from a true HIV-positive sample, RNase activity will be decreased using, for example, RNase A inhibitors (i.e. RNAsecure (Invitrogen), 5′8 diphosphoadenosine 3′-phosphate, Ribonucleoside Vanadyl Complex (NEB), and RNase OUT Recombinant Ribonuclease Inhibitor (Thermo Fisher)).

It is contemplated that some RNase A inhibitors will inhibit RNase A, but not Cas13a. As RNase A is not a HEPN-nuclease, its specific inhibitors are unlikely to inhibit the HEPN-nuclease of Cas13a dose-dependently. Briefly, dilutions of each ribonuclease inhibitor will be included into the assay both with and without Cas13a:crRNA RNP, the reporter oligonucleotide and isolated HIV virions of known concentration. Ribonuclease inhibitors that do not interfere with Cas13a activity will be tested directly in blood components mixed with reporter oligonucleotide with and without Cas13a:crRNAs to determine if background cleavage is minimized. Alternatively, samples will be heated to remove RNAse activity. Previous studies have shown that virions from other RNA viruses (Zika and Dengue) can be spiked into human serum and heated to 95° C. for 1-2 minutes to increase release of viral RNA for detection. It will be determined whether a heating step facilitates detection of HIV RNA and if it reduces background RNase, but not specific Cas13a, detection. Additional testing of other methods (e.g., mechanical or chemical lysis) may also be performed. All experiments will be optimized with spiked-in HIV virions into blood components containing our reporter and Cas13a:crRNA RNPs with or without heat pre-treatment. For all studies, read-out fluorescence will be monitored using CellScope and a plate reader as control.

### Example 4: Amplification of HIV RNA Before Testing

The Example illustrates that isolated HIV RNA can first be amplified, for example, using the bacteriophage-derived RNA-dependent RNA polymerase, Qβ replicase (see for example Shah et al (1994) J Clin Microbiol 32(11):2718).

HIV RNA was purified from Vsvg-pseudotyped virions containing HIV NL4-3with GFP in place of env. HIV RNA was incubated with nucleotides (NTPs) and with or without Qβ replicase in reaction buffer (100 mM HEPES-NaOH, pH 7.5; 10 mM MgCl₂, and 1 mM EDTA). Amplified RNA was first purified using phenol and then added to the HIV-Cas13a assay. In some instances, specified as “no cleanup,” the amplified mixture was directly added to the HIV-Cas13a assay.

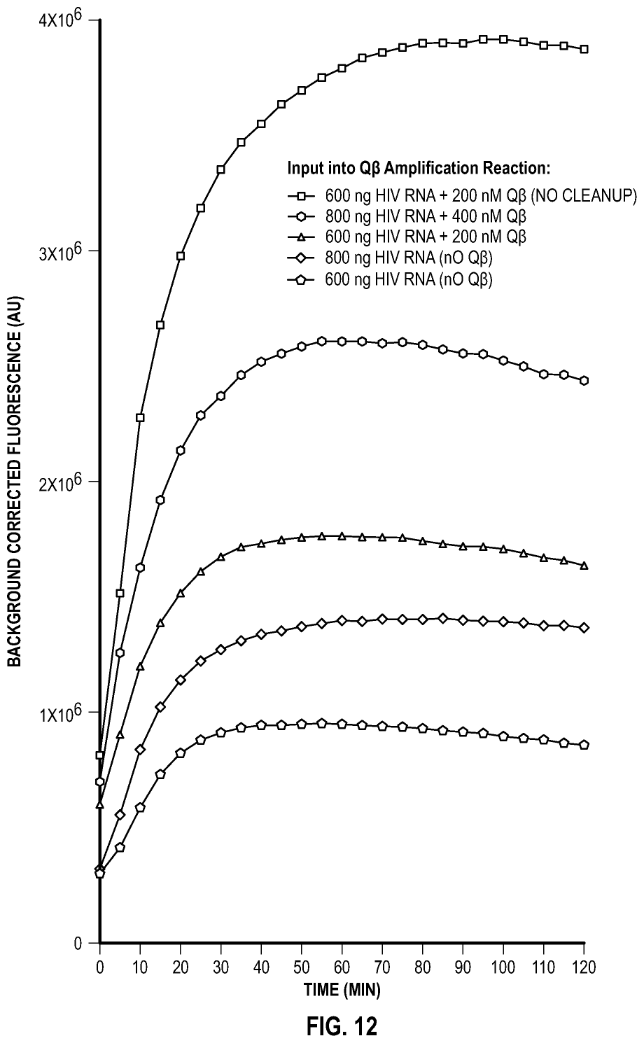

As shown in FIG. 12, Qβ replicase-amplified HIV RNA can provide improved sensitivity in the HIV-Cas 13a assay. No clean-up of the amplified product is needed before measuring the concentration of HIV RNA in the HIV-Cas13a assay.

### Example 5: Cas13a Detection of HCV Transcripts

CRISPR RNA guides (crRNAs) were designed for HCV. Nine crRNAs were designed for HCV. Each crRNA includes a crRNA stem that is derived from a bacterial sequence, while the spacer sequence is derived from the HCV genome (reverse complement). See Table 2 (reproduced below) for crRNA sequences.

TABLE 2
Examples of HCV crRNA Sequences

| SEQ | | |
|---|---|---|
| ID | | |
| NO: | Name | Sequence |
| 18 | PF016_crLbu_JFH1_1 | GACCACCCCAAAAAUGAAGGGGACUAAA |
| | | ACGUGUACUCACCGGUUCCGCA |
| 19 | PF017_crLbu_JFH1_2 | GACCACCCCAAAAAUGAAGGGGACUAAA |
| | | ACCCCUAUCAGGCAGUACCACA |
| 20 | PF018_crLbu_JFH1_3 | GACCACCCCAAAAAUGAAGGGGACUAAA |
| | | ACACCGGGUAGGUUCCCUGUUG |
| 21 | PF019_crLbu_JFH1_4 | GACCACCCCAAAAAUGAAGGGGACUAAA |
| | | ACGGGCGACCAGUUCAUCAUCA |
| 22 | PF020_crLbu_JFH1_5 | GACCACCCCAAAAAUGAAGGGGACUAAA |
| | | ACGACGAUGACCUUCUUCUCCA |
| 23 | PF021_crLbu_JFH1_6 | GACCACCCCAAAAAUGAAGGGGACUAAA |
| | | ACUUCCACUGCCAGUUGGAGCA |
| 24 | PF022_crLbu_JFH1_7 | GACCACCCCAAAAAUGAAGGGGACUAAA |
| | | ACGUUCAUCCAUUGGACCGCGC |
| 25 | PF023_crLbu_JFH1_8 | GACCACCCCAAAAAUGAAGGGGACUAAA |
| | | ACGGCUCGAGAAAGUCCAGAAC |
| 26 | PF024_crLbu_JFH1_9 | GACCACCCCAAAAAUGAAGGGGACUAAA |
| | | ACUCUGCAGAGAGACCAGUUAC |

Total RNA was isolated from Huh 7.5 cells infected with a MOI of 0.1 of the JFH-1 strain of HCV. Activator Huh 7.5 ssRNA targets were prepared at 100 μM in TE buffer, pH 8. RNA was calculated to 325 ng in 0.65 μL water containing diethyl pyrocarbonate (DEPC water).

A complex master mix was made by mixing 5× buffer, DEPC water, and crRNAs with SEQ ID NOs: 21 and 25 were diluted to 28 μM in TE buffer, pH 8, which was then combined with Cas13a protein. Controls without Cas13a protein were also made. The complex master mixes were then incubated at 37° C. for 30-60 minutes. DEPC water was added to lyophilized RNaseAlert (ThermoFisher Scientific) to resuspend. Target mixes were then made by adding RNase Alert, DEPC water, and 5× buffer to individually aliquoted Huh 7.5 RNA activator samples. Five μL of target mix was added to each well of a 384-well plate along with 15 μL of appropriate complex master mix. The formation of RNA cleavage products was monitored with a fluorometer.

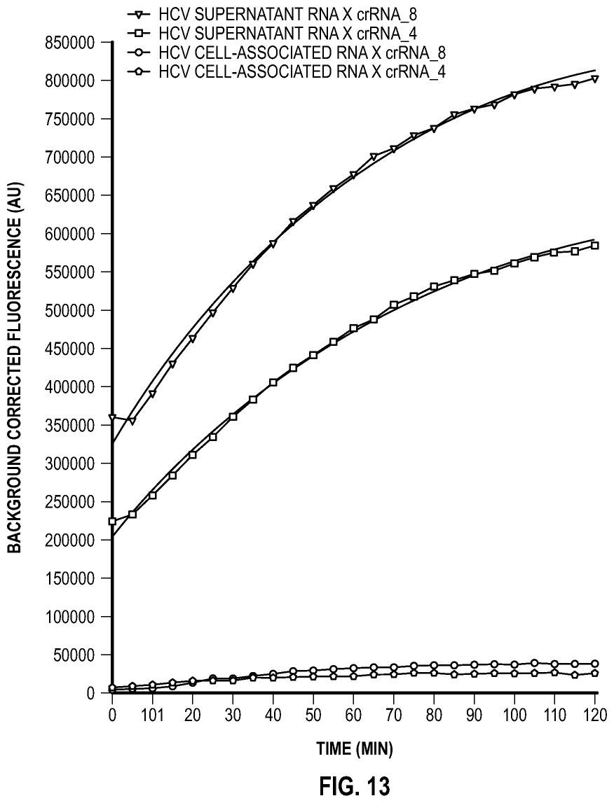

As shown in FIG. 13, cell-associated supernatant HCV RNA is detected robustly with both HCV-specific crRNAs (SEQ ID NO:21 or 25), compared to no detection with mock-treated cells (not shown).

### REFERENCES

East-Seletsky et al. Two distinct RNase activities of CRISPR-C2c2 enable guide-RNA processing and RNA detection, NATURE 538(7624): 270-273 (2016)
East-Seletsky et al. RNA Targeting by Functionally Orthogonal Type VI-A CRISPR-Cas Enzymes, MOLECULAR CELL 66(3): 373-383 (2017)

All publications, patent applications, patents and other references mentioned herein are expressly incorporated by reference in their entirety, to the same extent as if each were incorporated by reference individually. In case of conflict, the present specification, including definitions, will control.

The following statements provide a summary of some aspects of the inventive nucleic acids and methods described herein.

Statements:
1. A method for diagnosing the presence or absence of an HIV or HCV infection comprising:
   (a) incubating a sample containing RNA with a Cas13a protein and at least one CRISPR guide RNA (crRNA) for a period of time to form a RNA cleavage product; and
   (b) detecting a level of HIV or HCV RNA cleavage product with a detector,
   wherein the RNA is not reverse transcribed prior to the detecting step.
2. The method of statement 1, further comprising a step of amplification of the RNA and/or the HIV or HCV RNA cleavage products.
3. The method of statement 1, wherein the RNA and/or the HIV or HCV RNA cleavage product are not amplified.
3. The method of any one of the preceding statements, wherein the detector is a fluorescence detector, optionally a short quenched-fluorescent RNA.
5. The method of any one of the preceding statements, wherein the at least one crRNA is any one of SEQ ID NO: 1-8, 18-25 or 26.
6. The method of any one of the preceding statements, wherein the sample is incubated with 2, 3, 4, 5, 6, 7, 8, 9, 10 or more crRNAs.
7. The method of any one of the preceding statements, wherein the HIV or HCV RNA cleavage product concentration is determined using a standard curve and the level of the HIV or HCV RNA cleavage product.
8. The method of any one of the preceding statements, further comprising depleting a portion of the sample prior to detecting step.
9. The method of statement 8, wherein the portion of the sample is a human nucleic acid portion.
10. The method of any one of the preceding statements, further comprising removing RNase from the sample.
11. The method of statement 10, wherein RNase is removed from the sample using an

RNase inhibitor and/or heat.
12. The method of any one of the preceding statements, wherein the Cas13a protein and/or crRNA is lyophilized prior to incubation with the sample.
13. A method for quantifying HIV or HCV RNA concentration comprising:
   (a) incubating a sample containing RNA with a Cas13a protein and at least one CRISPR guide RNA (crRNA) for a period of time; and
   (b) analyzing the sample for HIV or HCV RNA cleavage product concentration with a detector,
   wherein the RNA is not reverse transcribed prior to the analyzing step.
14. The method of statement 13, wherein the HIV or HCV RNA cleavage product concentration is determined using a standard curve.
15. The method of any one of statements 14, wherein the HIV or HCV RNA cleavage product concentration is determined using a standard curve and the level of the HIV or HCV RNA cleavage product.
16. The method of statement 13 or statement 14, further comprising a step of amplification of the RNA and/or the HIV or HCV RNA cleavage product.
17. The method of any one of statements 13-16, wherein the RNA and/or the HIV or HCV RNA cleavage product are not amplified.
18. The method of any one of statements 13-17, wherein the detector is a fluorescence detector, optionally a short quenched-fluorescent RNA.
19. The method of any one of statements 13-18, wherein the at least one crRNA is any one of SEQ ID NO: 1-8, 18-25 or 26.
20. The method of any one of statements 13-19, wherein the sample is incubated with 2, 3, 4, 5, 6, 7, 8, 9, 10 or more crRNAs.
21. The method of any one of statements 13-20, further comprising depleting a portion of the sample prior to the analyzing step.
22. The method of statement 21, wherein the portion of the sample is a human nucleic acid portion.
23. The method of any one of statements 13-22, further comprising removing RNase from the sample.
24. The method of statement 23, wherein RNase is removed from the sample using an RNase inhibitor and/or heat.
25. The method of any one of statements 13-24, wherein the Cas13a protein and/or crRNA is lyophilized prior to incubation with the sample.
26. A method for identifying the presence or absence of HIV or HCV splice variants and/or mutations comprising:
   (a) incubating a sample containing RNA with a Cas13a protein and at least one CRISPR guide RNA (crRNA) for a period of time to form HIV or HCV RNA cleavage product; and
   (b) detecting the HIV or HCV splice variants and/or mutations by analyzing the HIV or HCV RNA cleavage product with a detector,
   wherein the at least one crRNA recognizes the HIV or HCV splice variants and/or mutations, and wherein the RNA is not reverse transcribed prior to the detecting step.
27. The method of statement 26, further comprising a step of amplification of the

RNA and/or the HIV or HCV RNA cleavage product.
28. The method of any one of statements 26-27, wherein the RNA and/or the HIV or HCV RNA cleavage product are not amplified.
29. The method of any one of statements 26-28, wherein the detector is a fluorescence detector, optionally a short quenched-fluorescent RNA.
30. The method of any one of statements 26-29, wherein the sample is incubated with 2, 3, 4, 5, 6, 7, 8, 9, 10 or more crRNAs.
31. The method of any one of statements 26-30, wherein the HIV or HCV RNA cleavage product concentration is determined using a standard curve and the level of the HIV or HCV RNA cleavage product.
32. The method of any one of statements 26-31, further comprising depleting a portion of the sample prior to the detecting step.
33. The method of statement 32, wherein the portion of the sample is a human nucleic acid portion.
34. The method of any one of statements 26-33, further comprising removing RNase from the sample.
35. The method of statement 34, wherein RNase is removed from the sample using an RNase inhibitor and/or heat.
36. The method of any one of statements 26-35, wherein the Cas13a protein and/or crRNA is lyophilized prior to incubation with the sample.
37. A method for monitoring reactivation or rebound of HIV or HCV transcription comprising:
   (a) incubating a sample containing RNA with a Cas13a protein and at least one CRISPR guide RNA (crRNA) for a period of time to form HIV or HCV RNA cleavage product; and
   (b) detecting an amount of the HIV or HCV RNA cleavage product in the sample with a detector.
38. The method of claim 1, further comprising a step of amplification of the RNA and/or the HIV or HCV RNA cleavage product.
39. The method of any one of statements 37-38, wherein the RNA and/or the HIV or HCV RNA cleavage product are not amplified.
40. The method of any one of statements 37-39, wherein the detector is a fluorescence detector, optionally a short quenched-fluorescent RNA.
41. The method of any one of statements 37-40, wherein the at least one crRNA is any one of SEQ ID NO: 1-8, 18-25 or 26.
42. The method of any one of statements 37-41, wherein the sample is incubated with 2, 3, 4, 5, 6, 7, 8, 9, 10 or more crRNAs.
43. The method of any one of statements 37-42, wherein the HIV or HCV RNA cleavage product concentration is determined using a standard curve and the level of the HIV or HCV RNA cleavage product.
44. The method of any one of statements 37-43, further comprising depleting a portion of the sample prior to the detecting step.
45. The method of statement 44, wherein the portion of the sample is a human nucleic acid portion.
46. The method of any one of statements 37-45, further comprising removing RNase from the sample.
47. The method of statement 46, wherein RNase is removed from the sample using an RNase inhibitor and/or heat.
48. The method of any one of statements 37-47, wherein the Cas13a protein and/or crRNA is lyophilized prior to incubation with the sample.
49. The method of any one of statements 1-48, further comprising treating a patient with a sample that has detectable HIV or HCV RNA.
50. The method of statement 49, wherein detectable HIV or HCV is at least 2 copies HIV or HCV/ml sample, at least 5 copies HIV or HCV/ml sample, or at least 10 copies HIV or HCV/ml sample, or at least 20 copies HIV or HCV/ml sample, or at least 30 copies HIV or HCV/ml sample, or at least 40 copies HIV or HCV/ml sample, or at least 50 copies HIV or HCV/ml sample.
51. A method comprising treating a patient with detectable HIV or HCV detected by a method comprising:
   (a) incubating a reaction mixture comprising an RNA sample from the patient with a Cas13a protein and at least one CRISPR guide RNA (crRNA) for a period of time sufficient to form a RNA cleavage product;
   (b) detecting a level of any HIV or HCV RNA cleavage product(s) that are in the mixture with a detector; and
   (c) treating a patent having detectable HIV or HCV in the sample with an HIV or HCV therapy.
52. The method of statement 51, wherein detectable HIV is at least 2 copies HIV/ml sample, at least 5 copies HIV/ml sample, or at least 10 copies HIV/ml sample, or at least 20 copies HIV/ml sample, or at least 30 copies HIV/ml sample, or at least 40 copies

HIV/ml sample, or at least 50 copies HIV/ml sample.
53. The method of statement 51, wherein detectable HCV is at least 2 copies HCV/ml sample, at least 5 copies HCV/ml sample, or at least 10 copies HCV/ml sample, or at least 20 copies HCV/ml sample, or at least 30 copies HCV/ml sample, or at least 40 copies HCV/ml sample, or at least 50 copies HCV/ml sample.
54. The method of any one of statements 49-52, wherein treating comprises administering to the patient one or more antiretroviral therapy (ART), combined antiretroviral therapy (cART), high active antiretroviral therapy (HAART), latency reversal agent (LRA), integrase strand transfer inhibitor (INSTI)-based regimen, regimen that includes a boosted protease inhibitor active against HIV-2 (e.g., darunavir or lopinavir), or a combination of HIV therapies.
55. The method of any one of statements 49-50 or 53, wherein treating comprises administering to the patient one or more of Daclatasir, Elbasvir-Grazoprevir, Glecaprevir-Pibrentasvir, Harvoni, Ledipasvir-Sofosbuvir, Ombitasvir-Paritaprevir-Ritonavir, Ombitasvir-Paritaprevir-Ritonavir and Dasabuvir, Peginterferon alfa-2a, Peginterferon alfa-2b, Ribavirin, Simeprevir, Sofosbuvir, Technivie, Sofosbuvir-Velpatasvir, Sofosbuvir-Velpatasvir-Voxilaprevir, Zepatier, or combinations thereof.
56. A kit comprising a package containing at least one Cas13a protein, at least one CRISPR guide RNA (crRNA), and instructions for detecting and/or quantifying HIV or HCV RNA in a sample (e.g., pursuant to the method of any of statements 1-55), where each of the CRISPR guide RNA(s) can have a sequence with about 70% or more sequence identity to any one of SEQ ID NO: 1-8, 18-25 or 26.
57. The kit of statement 56, further comprising at least one short quenched-fluorescent RNA.
58. The kit of statement 56 or 57, further comprising nuclease-free water, a buffer to regulate the pH of a solution, reaction vessel(s), and/or implements for collection of a sample from a patient.
59 The kit of statement 56, 57, or 58, further comprising a therapeutic agent for treatment of HIV or HCV infection.
60. A composition comprising one or more CRISPR guide RNA(s) comprising a sequence with at least 70% sequence identity to any one of SEQ ID NO: 1-8, 18-25 or 26.
61. The composition of statement 60, further comprising at least one Cas13a protein.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

In addition, where the features or aspects of the invention are described in terms of Markush groups, those skilled in the art will recognize that the invention is also thereby described in terms of any individual member or subgroup members of the Markush group.

What is claimed is:

1. A method comprising:
(a) incubating a sample containing RNA with a Cas13a protein and at least one CRISPR guide RNA (crRNA) for a period of time to form a RNA cleavage product; and
(b) detecting a level of HIV or HCV RNA cleavage product with a detector,
wherein the RNA is not reverse transcribed prior to the detecting step.
